(19) United States
(12) Patent Application Publication
(10) Pub. No.: US 2022/0134010 A1
(43) Pub. Date: May 5, 2022

(54) Testing Device and Method for Testing Moving Force of a Plunger of a Syringe

(71) Applicant: Lonza Ltd, Visp (CH)

(72) Inventors: Martin Vogt, Allschwil (CH); Roman Mathaes, Basel (CH); Atanas Koulov, Basel (CH); Hanns-Christian Mahler, Loerrach (DE)

(21) Appl. No.: 17/432,222

(22) PCT Filed: Feb. 21, 2020
(86) PCT No.: PCT/EP2020/054665

§ 371 Date: Aug. 20, 2021

Related U.S. Application Data
(60) Provisional application No. 62/812,319, filed on Mar. 1, 2019.

(30) Foreign Application Priority Data
Feb. 21, 2019 (EP) 19 158 665.0
Mar. 1, 2019 (EP) 19 160 308.3
Mar. 26, 2019 (EP) 19 165 158.7
Jun. 6, 2019 (EP) 19 178 775.3

(51) Int. Cl. A61M 5/315; G01K 1/02; G01L 5/00

(52) U.S. Cl. CPC A61M 5/31511; G01K 1/026; G01L 5/00; A61M 2205/3368; A61M 2205/332; A61M 2209/084; A61M 2205/70

(57) ABSTRACT

The present invention relates to a testing device for testing the moving force of a plunger of a syringe, comprising a fixture, FX, (3) for holding a syringe (8) in the testing device, TD, (1) for moving force testing, wherein the fixture (3) comprises an annular holding element, AHE, (5) for holding part of the syringe (8), and a support frame, SF, (30) having a support plate, SP, (300) for supporting the annular holding element, AHE, (5), wherein the annular holding element, AHE, (5) is detachably connected to the support plate, SP, (300) via an indirect connection via at least one intermediate element, IE, (10) between the at least one annular holding element, AHE, (5) and the support plate, SP, (300), wherein one intermediate element, IE, (10) is a tempering cylinder, TC, (7) for tempering at least part of the syringe (8). The invention further relates to a method for testing the moving force of a plunger of a syringe.

### FIELD OF THE INVENTION

The present invention relates to a testing device for testing the moving force of a plunger of a syringe as well as to a method for testing the moving force of a plunger of a syringe.

### TECHNOLOGICAL BACKGROUND

Syringes are typically used for administration of pharmaceuticals. For administration of pharmaceuticals so called prefilled syringes, PFS are preferably used. These PFS are filled with the respective pharmaceutical and are then transported and possibly stored.

Syringes typically comprise a barrel and a plunger. FIGS. 2 and 3 show two types of syringes. The two types of syringes 8 have in common that they comprise a barrel 80 and a plunger 81. The barrel 80 has a flange 801 at the back end and a shoulder 803 with a neck 804 at the front end. The lumen 800 of the barrel 80 extends between the back end and front end of the syringe 8. At the front end of the barrel 80 a dispensing opening 82 is provided. In the type of syringe 8 shown in FIG. 2 the dispensing opening 82 is provided by a needle cannula, NC, 820 which may also be referred to as staked needle, which is attached to the neck 804 of the barrel 80. In the type of syringe 8 shown in FIG. 3 the dispensing opening 82 at the neck 804 of the syringe 8 is formed by a Luer cone, LC, 821. Both types of syringes 8 depicted in FIG. 2 and FIG. 3 are intended to be used as PFS and therefor also comprise a syringe closure system, SCS, 83 for closing the dispensing opening 82 at the front end of the syringe 8. In the type of syringe 8 having a NC 820 at the front end, the SCS 83 is a needle shield and in the type of syringe having a Luer cone 821 at the front end, the SCS 83 is a tip cap.

The plunger 81 is inserted into the lumen 800 of the barrel 80 from the back end of the syringe 8. As shown in FIGS. 2 and 3 the plunger 81 may comprise a rod 810 and stopper 811. The stopper 811 being positioned on the distal end of the rod 810. The stopper 811 may be screwed onto the rod 810 or may be attached to the rod 810 in a different way. The stopper 811 may be made of silicone. The plunger 81, in particular the stopper 811, at the proximal side and the SCS 83 at the distal side seal the volume of the syringe 8, where the liquid to be administered is present.

If not defined differently, the term distal denotes the direction from the back end of the syringe to the front end of the syringe with the dispensing opening 82, and the term proximal denotes the opposite direction.

The plunger can be moved axially inside the lumen of barrel. By movement of the plunger in the barrel, the volume between the distal end of the plunger and the dispensing opening at the front end of the barrel can be decreased and any liquid in the syringe is ejected from the dispensing opening.

When moving the plunger in the barrel of the syringe, the plunger, in particular the stopper glides along the inner surface of the barrel.

In some embodiments, the syringe is provided with a casing. The syringe, for example, can be a passive safety guard syringe or an auto-injector. One embodiment of a passive safety guard syringe is schematically shown in FIG. 46b. One embodiment of the syringe is an auto-injector which is schematically shown in FIGS. 44b and 44c.

As the administration of pharmaceuticals should preferably be carried out in such a manner as to reduce any burden on the patient, it is desirable to ensure that the flow rate of liquids, in particular pharmaceuticals, which is injected into the body of the patient is constant and preferably at a low level. Furthermore, any irregularity during the injection should be avoided, such as different speeds of injection, but the injection should happen smoothly and uniformly.

The flow rate of liquids to be administered is influenced by various factors. One factor is the force which is applied to the plunger of the syringe. Another factor is the diameter of the dispensing opening.

The force which is necessary to move the plunger in the barrel will hereinafter be referred to as moving force of a syringe, moving force of a plunger of a syringe or simply as moving force. The moving force again is influenced by several factors. In particular, the force is influenced by the friction of the plunger with the inside of the lumen of the barrel. In addition, the moving force is influenced by the medium which is present in the volume of the barrel and by the barrel, plunger and needle or LC geometry and material.

In ISO 11040-4 (Third addition 2015-04.01 Prefilled syringes—Part 4: Glass barrels for injectables and sterilized subassembled syringes ready for filling) a method for measuring a glide force of a syringe is described (ISO 11040-4, Annex E). In this test, the barrel of the PFS is empty before testing. The empty syringe is placed between two adaptor plates which are appropriately sized for the barrel of the syringe such that the barrel is held in an opening formed by recesses at the edge of each adaptor plate (ISO 11040-4, Figure C.1).

This testing is disadvantageous in that adaptation of the testing device to the geometry of the syringe to be tested requires the adaptor plates to be exchanged. As these have to be attached to a force measurement instrument, which can also be referred to as a load frame, and their respective alignment in the load frame has to be ensured, the adaptation of the testing device to the geometry of the syringe is complex. In addition, this test only provides information on the glide force and thus only on one of the factors which influence the flow rate of liquids into the body of the patient or out of the body of the patient.

There was a need for a solution to allow reliable determination of the moving force necessary to move the plunger of syringes in the barrel of the syringe under different conditions, while the device used should be easy to handle.

Surprisingly, the problem was solved by providing devices and a method for testing the moving force, wherein the conditions of a syringe can easily be adjusted and the syringe can reliably be held during the testing.

### ABBREVIATIONS

PFS Prefilled syringes

SCS Syringe closure system

FX Fixture

SF support frame

SP support plate

CP carrier plate

AHE annular holding element

NC needle cannula

LC Luer cone

IE intermediate element

FH flange holder

NH needle holder

RC receiving cylinder

TC tempering cylinder

TD testing device

### SUMMARY OF THE INVENTION

Subject of the invention is a testing device for testing the moving force of a plunger of a syringe, comprising a load frame and a fixture, FX, for holding a syringe in the testing device, TD, for moving force testing, wherein the fixture comprises an annular holding element, AHE, for holding part of the syringe, and a support frame, SF, having a support plate, SP, for supporting the annular holding element, AHE, wherein the annular holding element, AHE, is detachably connected to the support plate, SP, via an indirect connection via at least one intermediate element, IE, between the at least one annular holding element, AHE, (5) and the support plate, SP, wherein one intermediate element, IE, is a tempering cylinder, TC, for tempering at least part of the syringe.

### DETAILED DESCRIPTION OF THE INVENTION

The testing device comprises a fixture, FX, for holding a syringe in a testing device, TD, for moving force testing, wherein the fixture comprises a support frame having a support plate and at least one annular holding element for holding part of the syringe, wherein the annular holding device is detachably connected to the support plate.

The TD comprises at least one holding unit. The holding unit is a fixture according to the invention. The TD further comprises a load frame for applying force onto the syringe. The support frame, SF, of the fixture is an open structure. The SF can have an upper plate and a lower plate as well as two poles connecting the upper and lower plate. The upper plate is preferably the support plate, SP, of the SF, which ultimately supports the syringe held in the annular holding element. The lower plate may serve for carrying a container for liquids and can thus also be referred to as carrier plate, CP. For this purpose, the lower plate may comprise a tray. The SF can comprise a foot for attaching the SF to the base of a load frame of the TD.

The annular holding element, AHE, serves for holding part of the syringe. For this purpose, the AHE has an axial through hole wherein a part of the syringe can be placed. The part of the syringe to be held in the AHE may be part of the barrel or the front end of the barrel comprising the dispensing opening, in particular a NC or LC.

Preferably, the testing device comprises more than one AHE.

By providing an AHE which is detachably connected to the SP of a SF, a number of advantages can be achieved. As the AHE can be detached from the SP and thereby from the fixture, it can be exchanged. For example, an AHE having a through hole of a first size may be replaced by an AHE having a through hole of a second size. Thereby, syringes of different sizes, for example with different diameters of the barrel or with different diameters of the NC can be held in the fixture. For changing the fixture from holding a syringe having a first size to a holding a syringe having a second size, only the AHE has to be replaced. Hence, the fixture is easy to be handled and can easily be adapted to the condition of the syringe, in particular to the size of the syringe.

The AHE is detachably connected to the SP via an indirect connection via at least one intermediate element IE between the at least one AHE and the SP. One IE is a TC for tempering at least part of the syringe.

According to one embodiment, the AHE has a head having a larger diameter than the body of the AHE and an opening of the IE has an inner diameter corresponding to the outer diameter of the body of the AHE. The AHE may be slid into the aperture and may be held by the head of the AHE which rests on the upper side of the IE at the circumference of the opening.

According to an embodiment, the indirect connection between the AHE and the SP is a screw-type connection. In this embodiment an opening in the IE can have an inner thread at the inner circumference and the AHE can have an outer thread. In this embodiment, the AHE may have a continuous outer diameter and the outer thread extends over the entire length of the AHE.

Alternatively, the AHE in this embodiment may also have a head and a body, with the diameter of the head being larger than the diameter of the body. In this case, the outer thread preferably extends over the length of the body of the AHE.

According to the invention, the connection between the AHE and the SP is an indirect connection. In particular, at least one intermediate element, IE, will be provided between the AHE and the SP. The IE may be a tempering element and/or a distance element. A tempering element according to the invention is an element which serves for heating or cooling at least part of the syringe. A distance element is an element which serves for maintaining a predetermined distance between the AHE and the SP or a further element of the FX such as a tempering element.

According to one embodiment the IE is a tempering cylinder, TC, a receiving cylinder, RC, or a spacer. As the connection between the AHE and the SP is an indirect connection, the AHE will be detachably attached to one of the IEs. The IE may be firmly attached to the SP or may be detachably attached to the SP. If more than one IE is present, the IEs may be firmly attached. It is, however, preferred that the IEs are detachably attached to each other. The firm attachment may be achieved by welding. The detachable attachment may be achieved by plug-type connection or screw-type connection.

According to one embodiment, the AHE is a flange holder, FH, for holding the flange of the syringe. In this embodiment, the through hole of the AHE has a diameter which at least at the upper end of the AHE is smaller than the outer diameter of the flange 801 of the syringe. The diameter of the through hole at the upper end of the FH corresponds to the outer diameter of the barrel of the syringe to be held in the fixture. The diameter of the through hole of the FH may be constant over its length. Preferably, the diameter of the through hole of the FH is, however, smaller at the upper end. That means that the diameter of the through hole is larger in the lower section of the FH than the diameter of the barrel. Preferably, the difference in diameters in the through hole is provided by a collar at the upper end of the through hole. The collar has a thickness of less than 10 mm, thereby complying with the dimensions prescribed in the ISO 11040-4.

According to an embodiment, the AHE is a needle holder, NH, for holding the needle cannula, NC, or a Luer cone, LC, of the syringe. In this embodiment, the diameter of the through hole of the AHE corresponds to the outer diameter of the NC or LC of the syringe to be held in the fixture.

According to one embodiment, the NH is a disc body. The outer diameter of the disc body preferably corresponds to an aperture in an IE.

According to an embodiment, the NH is a threaded bush. The threaded bush has an outer thread extending over its length, preferably over its entire length. The outer diameter preferably corresponds to the inner diameter of an IE, in particular a RC.

According to one embodiment, the AHE is a RC for receiving the barrel of the syringe to be held in the fixture. In this embodiment, the inner diameter of the through hole of the RC corresponds to the outer diameter of the barrel over at least part of the length of the through hole. The length of the RC is preferably longer than the length of the barrel of the syringe. Further preferably, the length of the RC is longer than the length of the barrel of the syringe with the NC or LC. The inner diameter of the through hole can be constant over the length of the RC. In a preferred embodiment, the inner diameter of the through hole of the RC is larger than the outer diameter of the flange in an upper section and corresponds to the outer diameter of the barrel in the lower section. In the upper section of the RC an outer thread may be provided. The outer thread may extend to the upper end of the RC. Alternatively, the RC may have a head with a larger diameter than the remaining part of the RC and the outer thread extends in the upper section of the RC from the lower end of the head.

According to one embodiment, the AHE has a two-part form.

According to one embodiment, one part of the AHE may be an upper part the AHE and the second part may be the lower part of the AHE. In this embodiment, the upper part of the AHE may be a FH and the lower part may be tubular element for receiving the barrel of the syringe and possibly also the NC or LC.

According to an alternative embodiment, one part of the AHE may be the outer part and the second part may be an inner part of the AHE. In that case, the inner part may be an insert made of a different material than the outer part. In particular, the AHE may be a NH, where the outer part is made of steel and the inner part or insert is made of bearing bronze. This embodiment is advantageous as the through hole for a NH has to have a very small diameter. Providing such a small diameter in a material having a lower hardness can be performed with a higher precision.

According to one embodiment, the fixture comprises at least two AHEs. The fixture may comprise a FH and a NH, a RC and a NH, a FH, a RC and a NH or two parts of a RC.

According to one embodiment, where the FX comprises at least two AHEs, one AHE is an FH and one is a NH.

According to one embodiment, where the FX comprises at least two AHEs, one AHE is a RC and one AHE is a NH.

According to one embodiment, where the FX comprises at least two AHEs, one AHE is a FH, one AHE is a RC and one AHE is a NH.

The at least two AHEs may be directly attached to one another. The attachment may be a screw-type connection or a plug-type connection. In one embodiment, the NH may be attached to the RC.

This attachment can be a screw-type attachment. In this embodiment, the NH preferably is a threaded bush which is screwed into the lower end of the through hole of the RC. In another embodiment, the at least two AHEs are an upper part and a lower part of a RC. The two parts may be connected via a screw-type connection.

Alternatively, the at least two AHEs may be indirectly attached via at least one IE. The attachment may be a screw-type connection or a plug-type connection. For example, a FH and a NH may be attached to a spacer. The spacer may be a bush or cylinder and the FH may be attached to the upper end of the bush for example by screw-type connection and the NH may be attached to the lower end of the bush for example by screw-type connection or by plug-type connection.

In case that there is only one IE, this one IE functions both as a tempering element and as a distance element.

In case that there are two or more IEs, the function of at least one of these IEs comprises the function of a tempering element; and the function of at least a second of these IEs comprises the function of a distance element.

Preferably, in case that there are two or more IEs at least one of these IEs functions as a tempering element; and at least a second of these IEs functions as a distance element;

More preferably, in case that there are two or more IEs, at least one of these IEs is a tempering cylinder, TC; and at least a second of these IEs is a spacer.

One embodiment of a distance element is a spacer (6) a different embodiment of the distance element is a RC.

One embodiment of a tempering element is a TC.

In the embodiment where the tempering element is a TC, the at least one AHE is preferably made of metal for transmitting heat to the syringe received in the TC. In a further preferred embodiment, the outer diameter of at least one AHE is in contact with the inner surface of a tempering room in the TC. Thereby, reliable heat transfer from the TC to the AHE and to the syringe can be ensured.

In one embodiment, the IE is a TC and the two AHEs are an upper part and a lower part of a two-part RC. The upper part may be screwed into the TC at an inner thread at the upper end of a tempering room and the lower part may be screwed into the TC at an inner thread at the lower end of the tempering room. The upper part can have a ring section with a tubular section extending from the bottom of the ring section and the lower part can have a ring section with a tubular section extending from the top of the ring section. The ring sections can have an outer thread provided on their outer circumference for screw-type connection with the IE, in particular with an inner thread at the TC. The tubular section of the RC is preferably in contact with the inner surface of the tempering room in the TC for transmitting heat from the TC to the RC over its entire length.

In one embodiment, an AHE is an RC and the RC is inserted into the tempering room of the TC. In this case, if a NH, in particular the threaded bush, is attached to the RC, in particular is threaded into the through hole of the RC, the temperature of the NC or LC can be adjusted according to the temperature set at the TC. If to the contrary, no NH, in particular no threaded bush is inserted into the RC, the NC or LC will not be in contact with the RC and the temperature of the NC or LC can thus not be adjusted via the TC. In this case, only the temperature of the barrel and the inside of the barrel can be adjusted by the TC via the RC, since the barrel will be in contact with the inner wall of the through hole of the RC.

According to one embodiment, the inner diameter of the upper part and the lower part of the two-part RC are different. In particular, the inner diameter of the lower part can be smaller or larger than the inner diameter of the upper part.

According to one embodiment, at least one step or a tapered section is provided at the inner diameter of the lower part of the two-part RC. With the at least one step or tapered section the diameter of the lower part decreases in the downward or distal direction. By providing one or more steps and or tapered section(s), the lower part can support or hold a surface of the syringe which is facing downwards, that means distally. In on embodiment, the surface of the syringe which is facing downward is a surface of a casing of the syringe.

According to one embodiment, at least one step or tapered section is provided at the inner diameter of the upper part of the two-part RC. With the at least one step or tapered section the diameter of the upper part decreases in the upward or proximal direction. By providing one or more steps and or tapered section(s), the upper part can support or hold a surface of the syringe which is facing upwards, i.e. proximally. In one embodiment, the surface of the syringe which is facing upwards is a surface of a casing of the syringe.

The casing may be the casing of an auto-injector syringe or the casing of a passive safety guard syringe.

According to one embodiment, the upper part of the two-part RC has an inner diameter which is at least smaller than the inner diameter of the lower part at its upper end. Thereby, a surface of the syringe, in particular of a casing of a syringe, facing upwards can come in contact with the bottom end of the upper part and can thus be held in the RC. In the lower part of this embodiment of the two-part RC at least one step is provided at the inner diameter. This step can be provided in the lower section of the lower part. The inner diameter above the step is larger than below the step. In particular the inner diameter above the step corresponds to or is equal to the outer diameter of the casing of the syringe and the inner diameter below the step is smaller than the outer diameter of the casing of the syringe. Thereby, a surface of the syringe, in particular of a casing of the syringe, facing downwards can be brought in contact with the step. In particular, the bottom of the casing can rest on the step. In one embodiment, the casing is the casing of an auto-injector syringe. In this case, the bottom part of the casing is a movable cap provided in a tubular casing. The movable cap can be referred to as a safety mechanism. In order to activate the auto-injector the movable cap first has to be pushed into the tubular casing. This force required for pushing the cap into the tubular casing can be applied by relative movement of the upper and lower part of the two-part RC. The relative movement can be effected by screwing the two parts into the IE, in particular into the TC.

According to one embodiment, the lower part of the two-part RC in addition to the step in the lower section has a tapered section over at least one part of the inner circumference. This tapered section can be a recess at the inner diameter extending from the top end of the lower part of the two-part RC in the distal direction. The recess can cover only part of the circumference of the inner diameter that means can extend over only a limited angle range. This recess can in particular be used for receiving a protrusion at the outer diameter of a casing of the syringe, for example at a window of the casing. In this embodiment, the inner diameter of the upper part of the two-part RC can be equal to or smaller than the inner diameter of the lower part at the top end in a section without the recess. Thereby, the upper surface of a protrusion of the casing can be brought in contact with the lower end of the upper part while the lower surface of the protrusion will be in contact with the bottom end of the recess. Thereby, an auto-injector syringe with side protrusions can be held between the upper part and the lower part of the two-part RC. The distance between the bottom end of the recess and the step of the lower part preferably corresponds to the distance between the bottom end of the cap of an auto-injector in the position where the cap has been moved into the tubular casing and the bottom end of the protrusion provided at the outer circumference of the tubular casing. The lower part of the two-part RC can be screwed into the IE, in particular TC, from the bottom and the auto-injector can be inserted into the lower part before the upper part of the two-part RC is inserted into the IE. Once the upper part is inserted and moved down by screwing movement, the top of the protrusion will initially rest against the bottom end of the upper part and the cap of the auto-injector will extend over the bottom end of the tubular casing. By further moving the upper part down in the IE, in particular by screwing, the step will push the cap into the tubular casing and thereby overcome the locking mechanism.

In another embodiment, the upper part has an inner diameter at the bottom section which is larger than the inner diameter of the upper section of the upper part. In particular, a step may be formed in the lower section of the upper part of the two-part RC. The lower part of the two-part RC in that case preferably has a tapered section at the inner diameter corresponding to the shield of a passive safety guard syringe. In this embodiment, the inner diameter in the upper section above the tapered section is larger than the inner diameter of the lower section below the tapered section. In addition the inner diameter in the upper section of the lower part is smaller than the inner diameter of the bottom section of the upper part of the two-part RC. In that embodiment, a flange of the shield of the passive safety guard syringe will be received in the larger inner diameter of the upper part of the two-part RC and will be supported downwards by the upper end of the lower part of the two-part RC.

The invention also relates to a fixture kit. According to one embodiment, a fixture kit can be provided, which comprises at least one SF and at least two AHEs. By providing more than one AHE, different conditions of the syringe can be accounted for. In particular, different sizes of the syringe and its parts, such as the NC and barrel can be considered during the testing.

According to one embodiment, the fixture kit comprises at least two AHEs, wherein each AHE has an axial through hole and the inner diameter of the at least two AHEs is different.

According to one embodiment, the fixture kit comprises at least one FH, at least one NH and at least one RC. By providing AHEs for different parts of a syringe, the respective parts can be positioned in different locations of the TD and may be treated differently during testing. For example, the different parts can be tempered to different temperatures.

According to one embodiment, the fixture kit comprises at least one IE. The IE is preferably a TC or a spacer. The IE can serve for connecting different AHEs. The TC and the spacer have an axial through hole. The axial through hole of the TC serves as a tempering room.

A further subject of the invention is a testing device, TD, for testing the moving force of a syringe, characterized in that the TD comprises a FX as defined herein, also with all its embodiments.

The TD comprises the FX. In addition, the TD can comprise a load frame for applying for onto the syringe in the FX and for measuring the force.

According to the invention, the FX of the TD comprises a TC.

According to one embodiment, the TC is firmly attached to the SP of a SF of the FX.

A further subject of the invention is a testing device, TD, for testing the moving force of a plunger of a syringe, characterized in that the TD comprises a TC for adjusting the temperature of a part of the syringe.

According to one embodiment, the TC is a TC for adjusting the temperature of only a part of the syringe.

Preferably, this TD comprises the TC and a holding unit for holding the syringe. The holding device is a FX as defined herein. In addition, this TD can comprise a load frame for applying for onto the syringe in the FX and for measuring the force. Preferably, the TC has at least one channel for transporting tempering medium through the TC. Preferably, the at least one channel is provided in the wall of the TC.

According to one embodiment, the TD comprises at least one heating and/or cooling unit for heating and/or cooling tempering medium. The heating and/or cooling unit is preferably a separate unit which is connected to the TC and in particular with the channel in the wall of the TC for fluid communication of the tempering medium with the TC.

According to one embodiment, the TC has a cylindrical wall enclosing a tempering room.

According to one embodiment, the TC is adapted for receiving only the barrel of the syringe in the tempering room. The TC can be adapted for receiving the specific part of the syringe by providing an AHE at the TC.

According to one embodiment, the TC is adapted for receiving an AHE in the form of an RC for receiving the barrel of the syringe inserted into the inner diameter of the TC.

According to one embodiment, the TC is adapted for receiving only the NC of the syringe in the tempering room. The TC can be adapted for receiving the specific part of the syringe by providing an AHE at the TC.

According to one embodiment, the TC is adapted for receiving an AHE which is a NH for receiving the NC of the syringe and an AHE which is a FH and the TD comprises a spacer.

According to one embodiment, the TC is adapted for receiving the barrel and the NC of the syringe in the tempering room. The TC can be adapted for receiving the specific part of the syringe by providing an AHE at the TC.

According to one embodiment, the TC has at least one attachment area for attaching an AHE. The attachment area is preferably located at the upper section of the inner diameter of the TC. As the inner diameter of the TC preferably limits the tempering room of the TC, the AHE can be attached at the tempering room and can ensure that a part of the syringe can be received in the tempering room.

According to one embodiment, the TD comprises a RC inserted into the inner diameter of the TC. In this embodiment, the at least the barrel of the syringe can be positioned in the tempering room of the TC and its temperature can be adjusted.

According to one embodiment, the TD comprises an AHE which is a NH and an AHE which is a RC and the NH is inserted into the lower section of the RC. In this embodiment, the barrel and the NC of the syringe can be positioned in the tempering room of the TC their temperature can be adjusted.

According to one embodiment, the TD comprises a TC and a spacer placed on the upper side of the TC.

According to one embodiment, the TD comprises an AHE which a NH and an AHE, which is a FH, and a spacer. In this embodiment, the NC of the syringe can be positioned in the tempering room of the TC and its temperature can be adjusted, while the barrel is outside of the tempering room of the TC. The NH is preferably attached to the TC. In particular, the NH is a disc body and is screwed into the upper section of the inner diameter of the TC. Preferably, the NH has a head with a larger diameter than the body of the NH. The NH, in particular the head of the NH is preferably received in the inner diameter of the spacer. The FH is preferably attached to the spacer in the upper area of the spacer. In particular, the FH is screwed into the inner diameter of the spacer.

According to one embodiment, the TD comprises at least two temperature sensors and the at least one AHE has at least two measuring holes for insertion of a temperature sensor. Preferably, the TD comprises three temperature sensors and the AHE has two measuring holes. In this embodiment, one temperature sensor can be used for measuring and outputting the actual temperature in the vicinity of a part of the syringe. The second temperature sensor can be used for measuring the actual temperature in the vicinity of a part of the syringe and transmitting the measured temperature to a cooling/heating unit. The first and the second temperature sensor are inserted into the respective measuring holes. The third temperature sensor can be used for measuring the temperature inside of the syringe, in particular in the barrel of the syringe.

According to one embodiment, the load frame is a load frame for applying pressure on the plunger of a syringe held in the FX of the TD and for measuring the force for moving the plunger of the syringe.

A further subject of the invention is a method for testing the moving force of a plunger of a syringe. The method is characterized in that it is carried out with a TD according to the invention.

According to one embodiment the method comprises the steps
a) setting the temperature in at least part of the syringe via the tempering cylinder, TC
b) applying a pressure on the plunger of the syringe via a load frame
c) measuring the force for moving the plunger in the syringe via the load frame.

The part of the syringe where the temperature is set, can be either the NC or the barrel or the barrel and the NC. The temperature in the respective part(s) of the syringe is preferably set by TC which is connected to the cooling/heating unit.

According to one embodiment, the method comprises step
d) monitoring the temperature present at at least part of the syringe via at least one temperature sensor.

The monitoring can be carried out by using at least one temperature sensor inserted into measuring holes of an AHE or by using a temperature sensor inserted into the barrel of the syringe.

According to one embodiment, step

e) comprises monitoring the temperature in the vicinity of the barrel, in the vicinity of the NC or LC or at the inside of the barrel.

According to one embodiment, the method comprises the step
f) adjusting the temperature of at least part of the syringe.

Adjusting the temperature of at least part of the syringe means that the temperature, which was set before, is changed in that at least one part of the syringe. The temperature can be adjusted via the TC.

According to one embodiment, the method is carried out with a fixture kit.

### BRIEF DESCRIPTION OF THE DRAWINGS

The present invention will be described again with reference to the enclosed drawings, wherein:

FIG. 1: shows a schematic perspective view of a first embodiment of the fixture according to the invention;

FIG. 2: shows a schematic view of one type of a syringe, syringe with a NC and a needle shield;

FIG. 3: shows a schematic view of another type of a syringe, syringe with a LC and a tip cap;

FIG. 4: shows a perspective view of the upper components of the embodiment of the fixture of FIG. 1;

FIG. 5: shows a perspective view of the upper components of the embodiment of the fixture of FIG. 1;

FIG. 6: shows a perspective view of the lower components of an embodiment of the fixture according to the invention;

FIG. 7: shows a perspective view of a second embodiment of the fixture according to the invention;

FIG. 8: shows a schematic sectional view of the support frame of the second embodiment of the fixture according to FIG. 7;

FIG. 9: shows a schematic view of an embodiment of the fixture kit according to the invention;

FIG. 10: shows a schematic sectional view of a first embodiment of a testing device according to the invention;

FIGS. 11, 12, 13: show perspective views of a first embodiment of a receiving cylinder of the testing device according to FIG. 10;

FIGS. 14, 15: show perspective bottom views of a first embodiment of a receiving cylinder of the testing device according to FIG. 10;

FIGS. 16, 17: show perspective views of the first embodiment of a testing device according FIG. 10 during assembly;

FIG. 18: shows a perspective view of the first embodiment of a testing device according FIG. 10 after assembly;

FIG. 19: shows a perspective bottom view of the first embodiment of a testing device according FIG. 10 after assembly;

FIGS. 20 and 21: shows perspective views of embodiment of temperature sensors of the testing device according to the invention;

FIG. 22: shows a schematic sectional view of a second embodiment of a testing device according to the invention;

FIGS. 23, 24, 25: show perspective views of receiving cylinder with needle holder of the second embodiment of the testing device according to FIG. 22;

FIG. 26: shows a sectional view of the needle holder of the second embodiment of the testing device according to FIG. 22;

FIGS. 27, 28: show perspective views of the second embodiment of the testing device according FIG. 22 during assembly;

FIG. 29: shows a schematic sectional view of a third embodiment of a testing device according to the invention;

FIGS. 30, 31: show perspective views of a needle holder of the third embodiment of the testing device according FIG. 22 during assembly;

FIG. 32: shows a perspective view of the needle holder of the third embodiment of the testing device according FIG. 22;

FIG. 33: shows a perspective view of the third embodiment of the testing device according FIG. 22 with a syringe;

FIGS. 34-37: show perspective views of the third embodiment of the testing device according FIG. 22 during assembly;

FIGS. 38, 39: show perspective views of the third embodiment of the testing device according FIG. 22 after assembly;

FIGS. 40-43: show graphs of moving force values

FIG. 44a: show a schematic sectional view of a fourth embodiment of the testing device;

FIGS. 44b and 44c: show schematic side views of an auto-injector for a syringe.

FIG. 45a: shows a schematic sectional view of a fifth embodiment of the testing device;

FIG. 45b shows a schematic side view of an embodiment of an auto-injector syringe;

FIG. 46a shows a schematic sectional view of a sixth embodiment of the testing device with a passive safety guard; and

FIG. 46b shows a schematic perspective view of an embodiment of a passive safety guard syringe.

### DETAILLED DESCRIPTION OF PREFERRED EMBODIMENTS

The present invention will now be described in more detail with reference to the enclosed figures. Same components and arrangements are denoted in the figures by the same reference numerals and the respective description may be omitted in order to avoid redundancies.

Features and advantages which are described with respect to the fixture also apply to fixture kit, the testing devices and the method for testing and vice versa and are only described once.

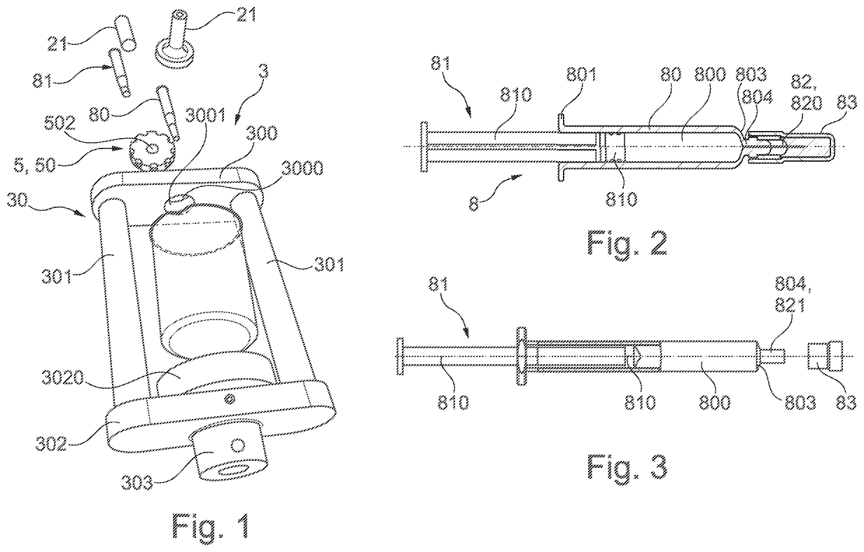

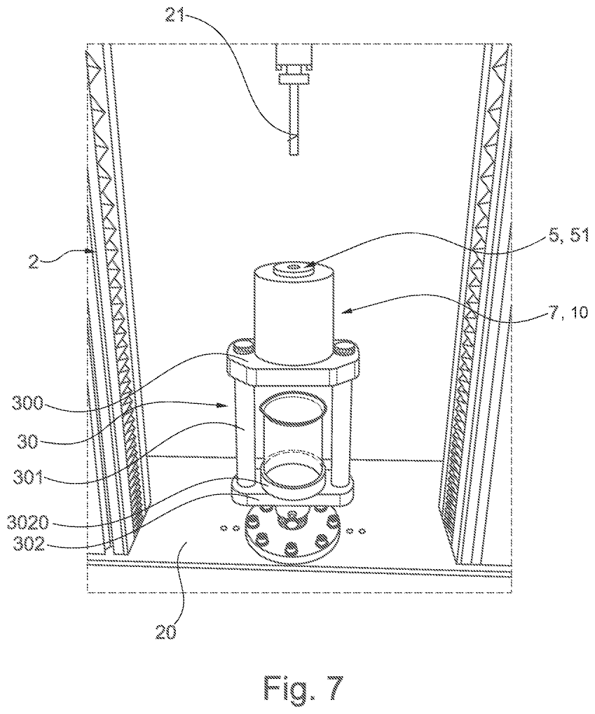

In FIG. 1 a schematic perspective view of a first embodiment of the fixture 3 according to the invention is shown. In the depicted embodiment, the fixture 3 comprises a SF 30. The SF 30 consists of a SP 300, a CP 302 and two poles 301 extending therebetween. The poles 301 can be firmly attached to the SP 300 and the CP 302 for example by welding, or they can be attached by a screw-type connection. The SP 300 is the upper plate of the SF 30 and the CP 302 is the lower plate. On the CP 302 a tray 3020 is mounted. The tray 3020 has a circular shape and a circular rim for holding a beaker. At the bottom of the CP 302 a foot 303 extends downwardly. In the SP 300 an aperture 3000 is provided in the middle. The aperture 3000 has an inner thread 3001. The aperture 3000 serves for receiving an AHE 5. In the first embodiment of the fixture 3 shown in FIG. 1, the AHE 5 is a FH 50. In FIG. 1 only the head 501 of the FH 50 can be seen. At the outer circumference of the head 501 recesses are provided for facilitating gripping of the head 501 and screwing of the FH 50. The FH 50 has a through hole 502 extending over its entire height. The through hole 502 serves for receiving the barrel 80 of a syringe 8 to be held in the fixture 3. The syringe 8 can have the design as shown in FIG. 2 as described herein. The plunger 81 of the syringe 8 will be moved in the TD 1, which will be described later, by a post 21, which can be attached to a load frame 2 (see FIG. 7).

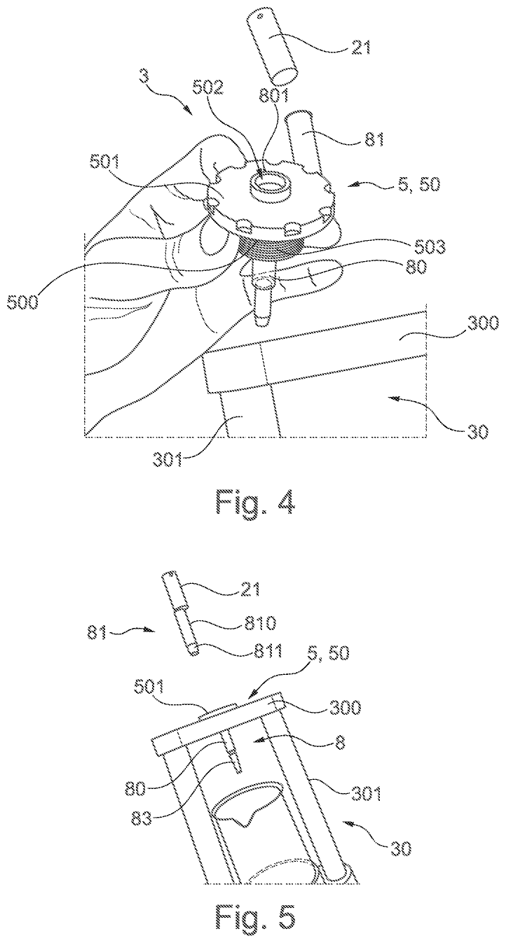

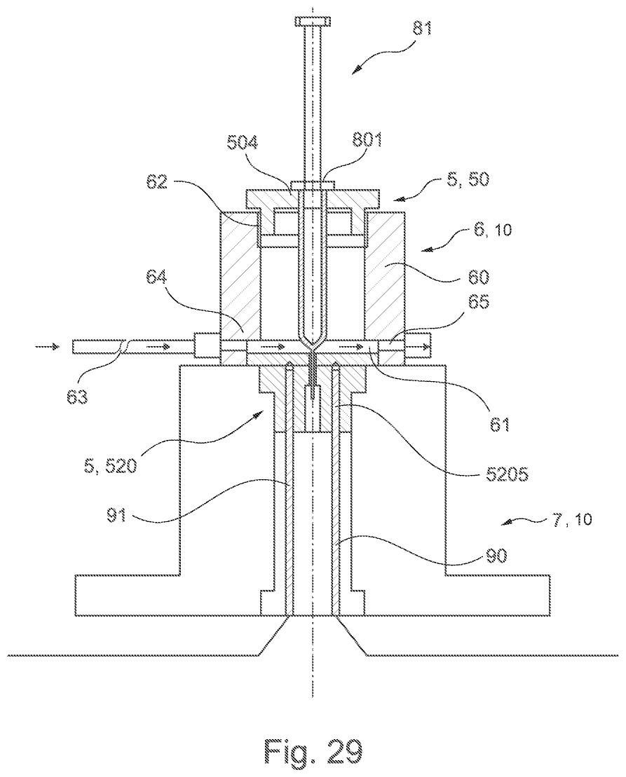

In FIG. 4 the FH 50 is shown with the barrel 80 of the syringe 8 inserted into the through hole 502 of the FH 50. The FH 50 has a body 500 extending downward from the head 501. The body 500 has a smaller out diameter than the head 501. The inner diameter of the body 500 can be larger than the through hole 502 at the head 501. Thereby a collar 504 (see FIG. 29) is formed. An outer thread 503 is provided on the outer circumference of the body 500. The height of the body 500 corresponds to the thickness of the SP 300. As can be derived from FIG. 4, the flange 801 of the barrel 80 rests on the upper side of the head 501 of the FH 50 while the barrel 80 extends through the through hole 502 and protrudes over the bottom end of the FH 50, in particular the bottom end of the body 500 of the FH 50.

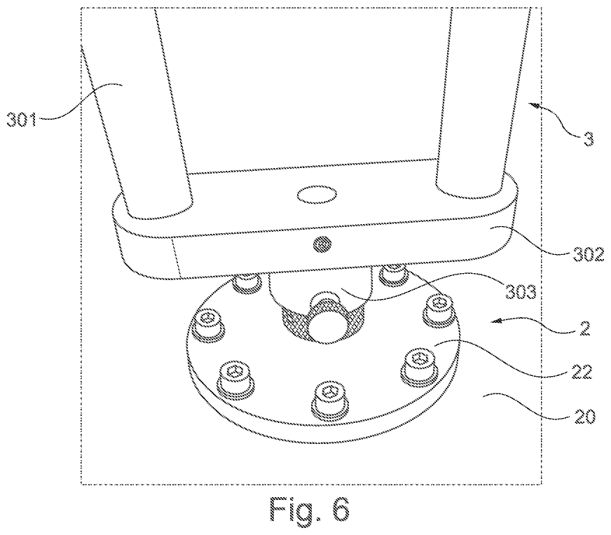

FIG. 5 shows the FX 3 with the FH 50 screwed into the aperture 3000 of the SP 300 and with the barrel 80 of the syringe 8 inserted into the through hole 502 of the FH 50. In this state, the plunger 81 can be inserted into the barrel 80 and the SCS 83 can be removed. Once the FX 3 is mounted in a load frame 2, the plunger 81 can be pushed down by the post 21. The force necessary for moving the plunger 81 will be measured. If the syringe 8 is a PFS, the liquid contained in the volume of the syringe 8 will be dispensed via the dispensing opening 82 of the syringe 8 into the beaker sitting on the tray 3020 of the fixture 3.

FIG. 6 shows how the FX 3 can be attached to the load frame 2. The foot 303 of the SF 30 is attached to a protrusion of a connecting disc 22. The protrusion is arranged in the middle of the connecting disc 22. The connecting disc 22 is attached to the base 20 of the load frame 2 by screws.

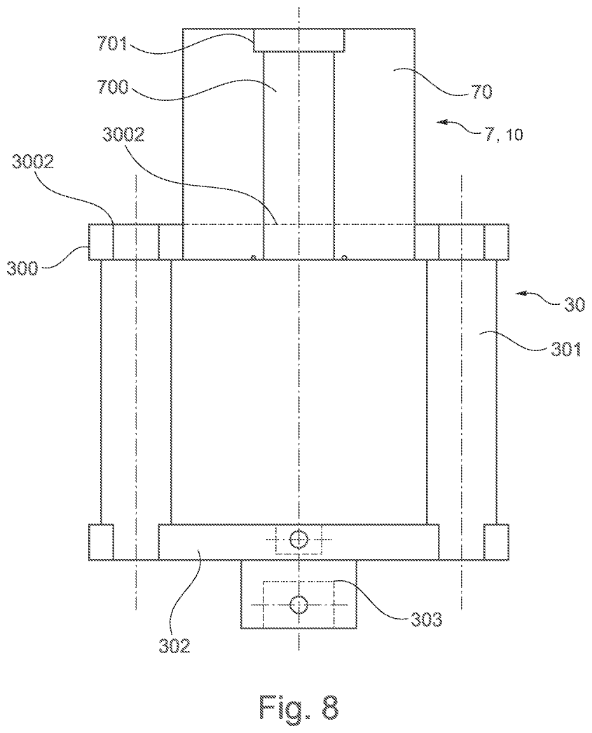

In FIG. 7 a second embodiment of the FX 3 according to the invention is shown. The second embodiment differs from the first embodiment in that the SP 300 is attached to the poles 301 of the SF 30 by screws. In addition, in the second embodiment, an IE 10, in particular a TC 7 is mounted to the SP 300. The TC 7 may be attached to the top side of a SP 300 of FIG. 1. This position of the TC 7 is indicated in FIG. 8 with the dashed line. The tempering room 700 within the TC 7 in that case is aligned with the aperture 3000 of the SP 300. Preferably, the TC 7 is, however, received in a large mounting opening in the SP 300 and extends through the mounting opening with the bottom of the TC 7 being in the same plane as the bottom side of the SP 300 (see FIG. 8). As will be described in further detail below, the second embodiment of the FX 3 also differs from the first embodiment by the AHE 5 used to hold the syringe. The AHE 5 of the second embodiment is a RC 51. The RC 51 is attached to the TC 7 by insertion of the RC 51 into the tempering room 700 of TC 7. Thereby an indirect connection between the AHE 5 and the SP 300 is provided with IE 10 which is the TC 7.

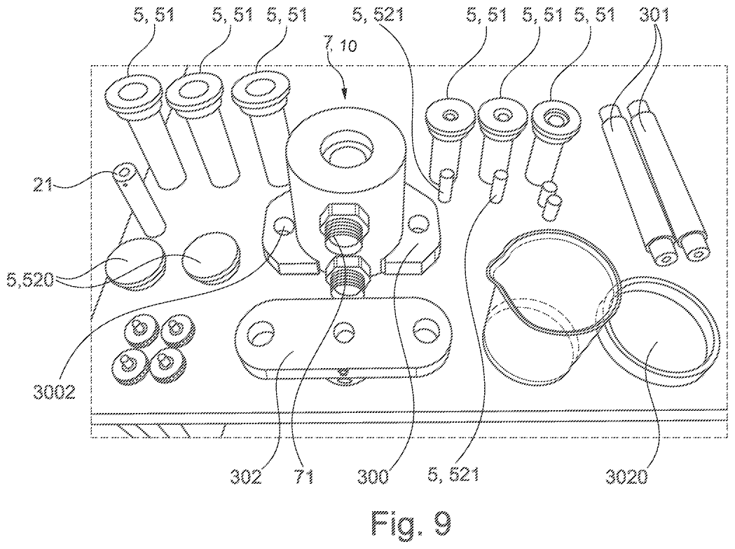

As can be derived from FIG. 8 which shows a schematic sectional view of the support frame 30 of the second embodiment of the FX 3, the TC 7 has an annular wall 70. The annular wall 70 encloses the tempering room 70 which extends over the entire height of the TC 7. In the wall 70 a channel (not shown) for tempering medium is provided. The tempering medium is provided to the TC 7 via a connector 71 (see FIG. 9) and exits from the TP 7 via another connector 71. The temperature of the tempering medium is set and can be adjusted at a heating/cooling unit (not shown). The TC 7 is connected to the heating/cooling unit preferably via tubes which are attached to the connectors 71. At the upper section of the tempering room 700 an inner thread 701 is provided at the circumference. The inner diameter of section with the inner thread 701 is larger than the inner diameter of the tempering room 700.

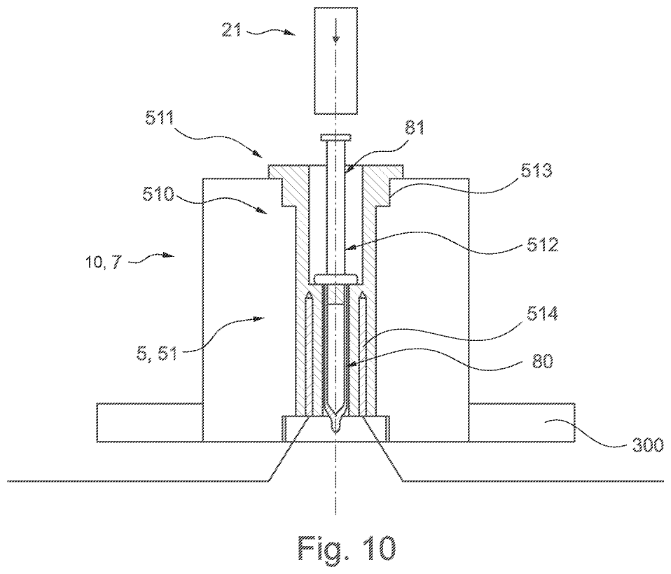

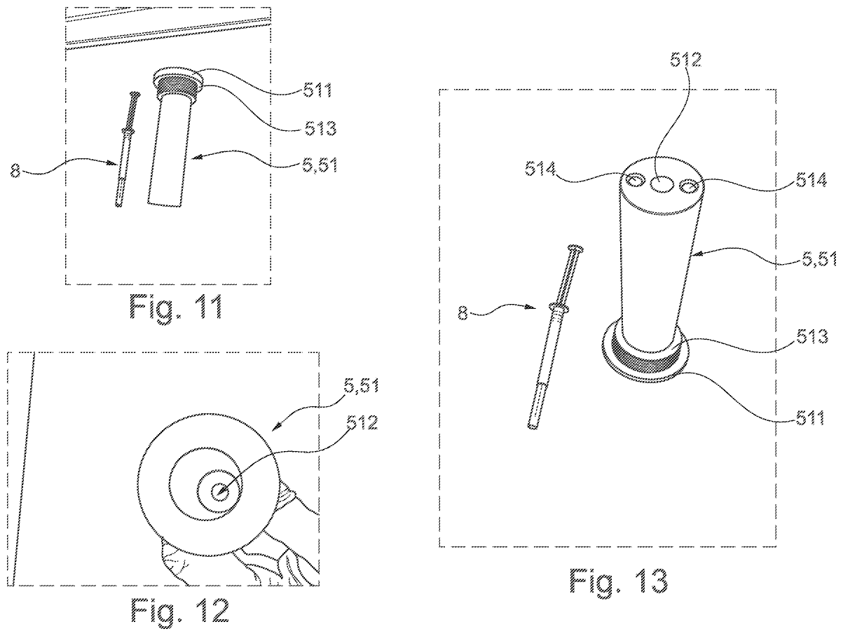

In FIG. 10 a first embodiment of a TD 1 according to the invention is shown. In FIG. 10 only the TC 7 with the AHE 5 and the SP 300 of the TD 1 is shown. In this first embodiment, an AHE 5 which is a RC 51 is inserted into the tempering room 700. As can be seen in FIG. 11, the RC 51 has a head 511 and a body 510 extending from the bottom of the head 511. The head 511 has a larger diameter than the body 510. At the upper section of the body 510 an outer thread 513 is provided. The section with the outer thread 513 has a larger diameter than the remaining part of the body 510. The outer diameter of the section of the body 510 with the outer thread 513 corresponds to and preferably is equal to the inner diameter of the section of the tempering room 700 with the inner thread 701. The outer diameter of the part of the body 510 without the outer thread 513 corresponds to the inner diameter of the tempering room 700 and preferably is equal to the inner diameter of the tempering room 70. Thereby, the outside surface of the part of the body 510 of the RC 51 without the outer thread 513 is in close contact with the inner side of wall 70 of the TC 7 and the temperature set by the tempering medium in the wall 70 of the tempering room 700 of the TC 7 can be conveyed to the RC 51.

The length of the RC 51 corresponds to and preferably is equal to the height of the TC 7.

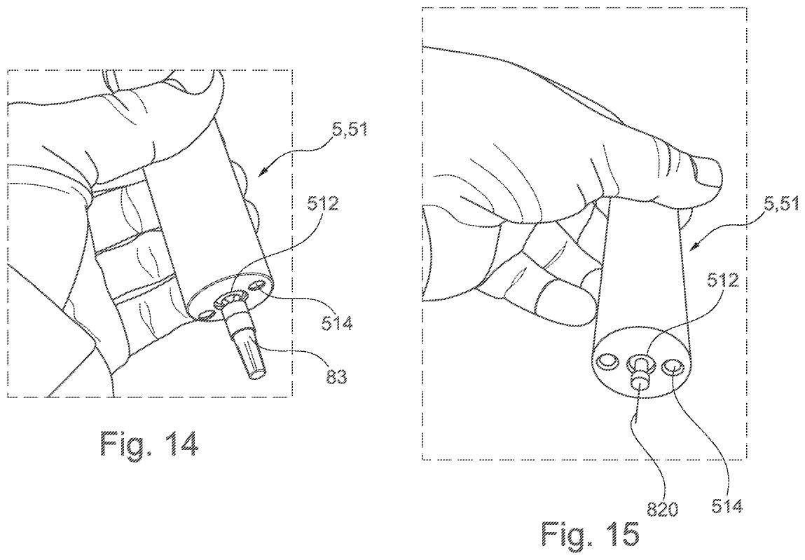

The RC 51 has a through hole 512. The inner diameter of the through hole 512 in the upper section is larger than in the lower section. This can be best seen in FIG. 12. In the upper section the plunger 81 of the syringe 8 to be held in the FX 3 of the TD 1 is located. The inner diameter of the upper section is larger than the diameter of the post 21 of the load frame 2, which is to apply force onto the plunger 81. The length of the lower section corresponds to the length of the barrel 80 of the syringe 8 to be held in the fixture 3 of the TD 1. The inner diameter of the lower section of the through hole 512 corresponds to the outer diameter of the barrel 80 and preferably is equal to the outer diameter of the barrel 80. The flange 801 of the barrel 80 rests on the step 516 formed by the different diameters of the upper and the lower section of the through hole 512. The NC 820 extends over the bottom end of the through hole 512. This is shown in FIGS. 14 and 15.

In the RC 51 of this embodiment two measuring holes 514 are provided. The measuring holes 514 extend from the bottom end of the RC 51 in the axial direction. The position of the measuring holes 514 can be best seen in FIG. 13. The length of the measuring holes 514 correspond to the length of the lower section of the RC 51. The measuring holes 514 are parallel to the through hole 512 and in the mounted condition of the RC 51 in the TC 7 are positioned between the inner side of the wall 70 and the through hole 512 of the RC 51. Temperature sensors (not shown) are inserted into the measuring holes 514 from the bottom.

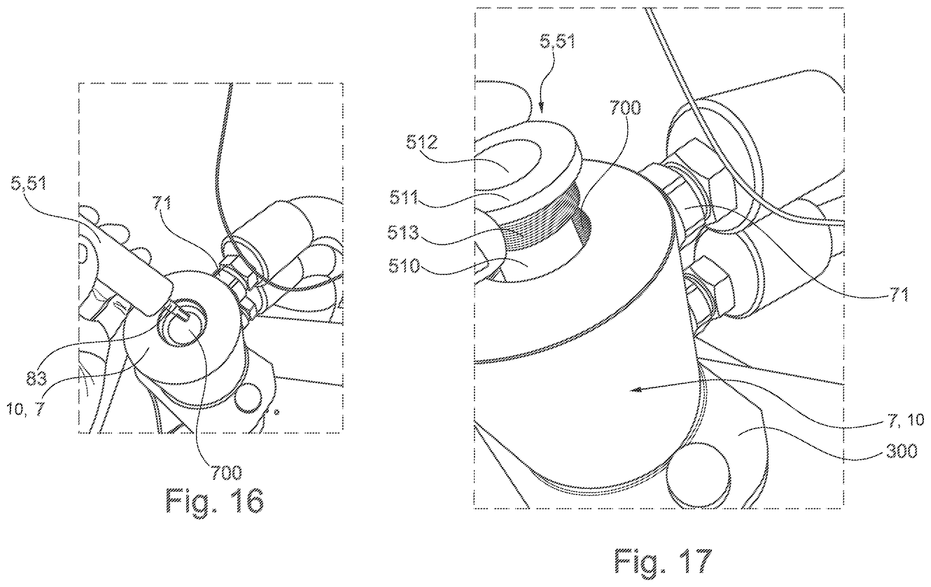

The RC 51 can be inserted into the tempering room 700 of the TC 7 as shown in FIGS. 16 and 17. In these figures, the syringe 8 is already inserted into the RC 51. Preferably, the syringe 8 will however only be inserted into the RC 51 once it is inserted and attached to the TC 7. Once the RC 51 is received in the tempering room 700 it will be attached to the wall 70 of the TC 7 by screw-type connection between the outer thread 513 of the RC 51 and the inner thread 701 of the TC 7.

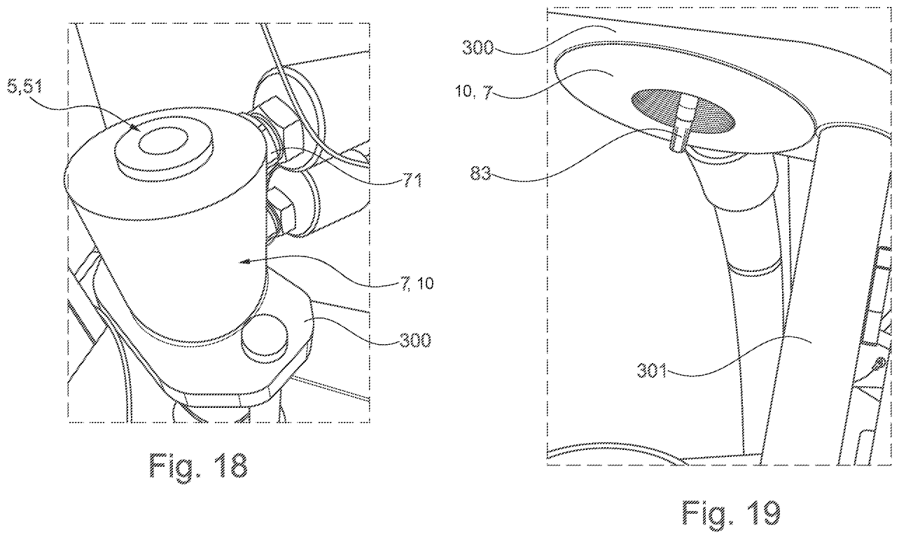

FIG. 18 shows the RC 51 in the mounted position in the TC 7. As can be derived from FIG. 19, the NC 820 which is covered in FIG. 19 with the SCS 83, extends over the bottom surface of the TC 7.

With the first embodiment of the TD 1 as shown in FIGS. 10 to 19 the temperature of the barrel 80 of the syringe 8 can be set and adjusted by the TC 7. The temperature and any temperature changes can be monitored by sensors which are introduced into the measuring holes 514 of the RC 51.

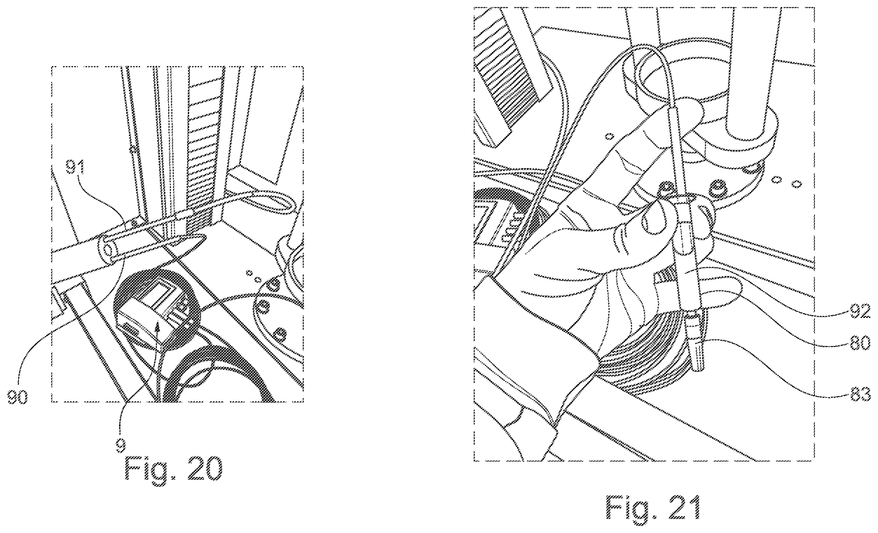

FIGS. 20 and 21 show a sensing unit 9 and sensors 90, 91, 92 to be used in the TD 1. In FIG. 20 two sensors 90, 91 have been inserted into the measuring holes 514 of the RC 51. In FIG. 21, a third sensor is introduced into the barrel 80 of the syringe 8. By using three sensors 90, 91, 92, the temperature of the barrel 80 and of the inside of the barrel 80 can be measured, verified and adjusted. In particular, a first sensor 91 is connected to the heating/cooling unit and serves for controlling the heating/cooling unit. Second sensor 90 is an external sensor, that means is not connected to the heating/cooling unit. The second sensor 90 servers for measuring the actual temperature and thus for confirming that the temperature set at the heating/cooling unit is reached at the RC 51. The third sensor 92 serves for measuring the temperature within the barrel 80. In particular, the third sensor serves for measuring the temperature of a liquid in the barrel. By using this third sensor, any difference between the temperature measured by the second sensor 91 and the third sensor 92 can be taken into account when measuring the temperature during a moving force test with the TD 1, and can also be used for adjustment of the temperature of the heating/cooling unit.

These sensors 90, 91, 92 can be used for any embodiment of the TD 1.

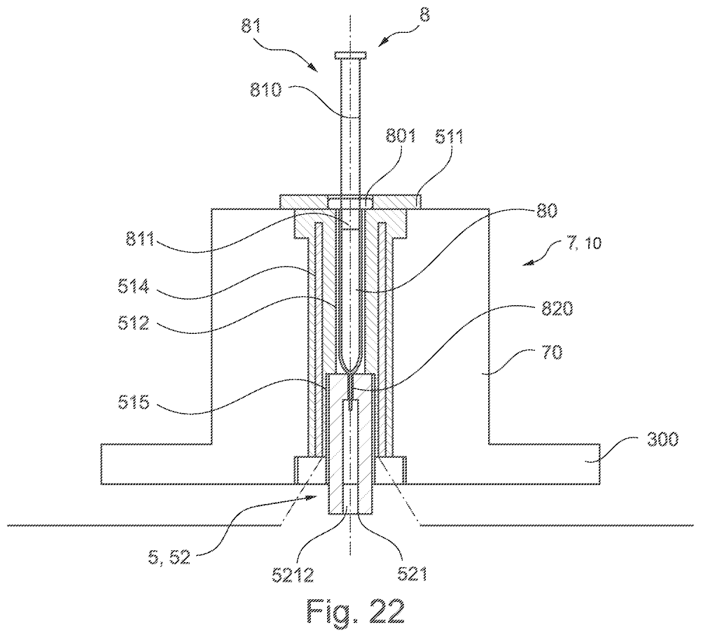

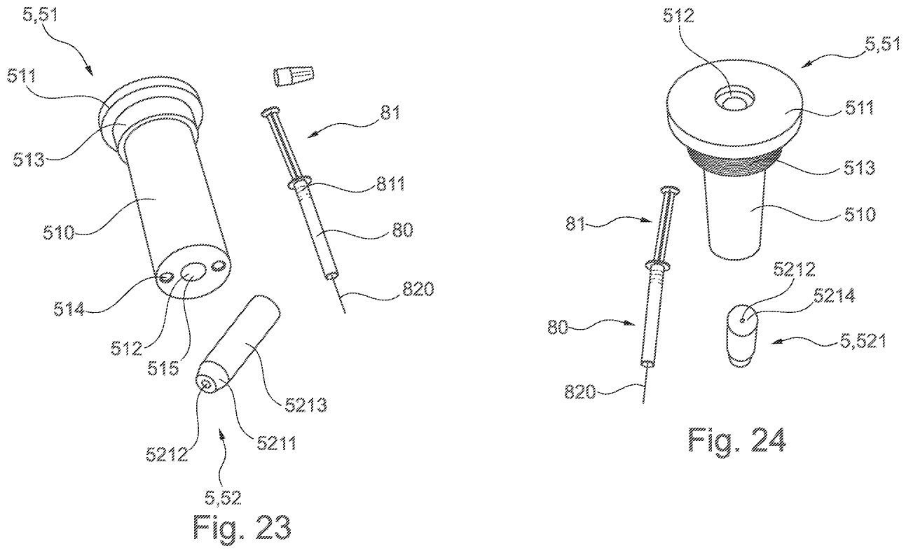

In FIG. 22 a second embodiment of the TD 1 is shown. In FIG. 22 only the TC and the AHEs 5 of the TD 1 are shown. This embodiment differs from the first embodiment by the layout of the RC 51. The RC 51 of the second embodiment differs from the RC 51 of the first embodiment as shown in FIGS. 10 to 19 only in that the inner diameter of the through hole 512 is continuous over the length of the through hole 512 and that a further AHE 5 is inserted into the bottom of the RC1 via screw-type connection. For this purpose, an inner thread 515 is provided at the lower section of the through hole 512 of the RC 51.

The inner diameter of the through hole 512 of the RC 51 of the second embodiment corresponds to and preferably is equal to the outer diameter of the barrel 80 of the syringe 8 to be held in the TD 1.

The additional AHE 5 which is inserted into the through hole 512 from the bottom is a NH 52 in the form of a threaded bush 521. The threaded bush 521 has a body 5210 and a head 5211, the latter is in the mounted position at the bottom of the threaded bush 521. The body 5210 has an outer thread 5213. The outer diameter of the body 5210 corresponds to the inner diameter of the lower part of the through hole 512 of the RC 51. The threaded bush 521 has a through hole 5212, which extends over the entire length of the threaded bush 521.

As can be best seen in FIG. 26, which shows the end of the body 5210 of the threaded bush 521 opposite to the head 5211, the inner diameter of the through hole 5212 is smaller at that end than over the remaining part of the body 5210. In particular, at the end opposite the head 5211, an insert 5214 in form of a bush is provided in the threaded bush 521. The insert 5214 has a cylindrical shape, wherein the inner diameter of the insert 5214 forms the through hole 5212 and the outer diameter is received in a recess 5215 provided in the end of the threaded bush 521 opposite the head 5211. The inner diameter of the insert 5214 corresponds the outer diameter of the NC 820. Here the NC 820 is received. These two equal diameters provide for a transmission of the temperature of the TC 7 to the NC 820. If no threaded bush is inserted into the RC 51, only the temperature of the barrel which is received in the RC 51 can be adjusted. The inner diameter of the threaded bush 521 in the section adjacent to the recess 5215 in the direction towards the head 5211 is smaller than the diameter of the recess 5215. Thereby a step is formed and the axial end of the insert 5214 which is received in the recess 5215 abuts on the step.

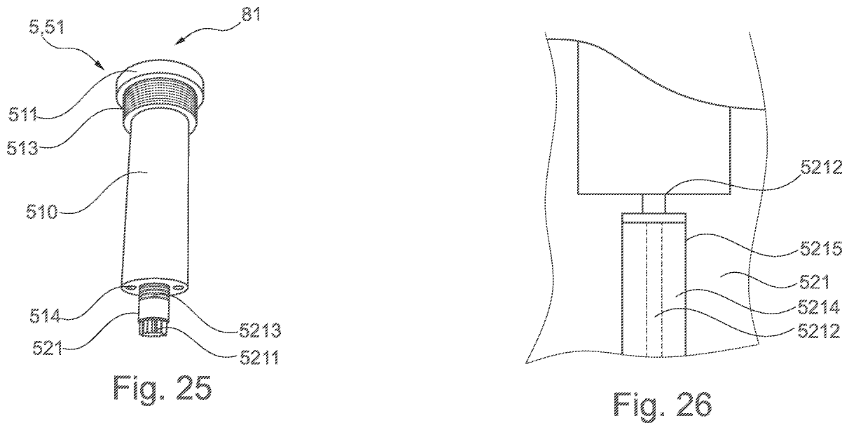

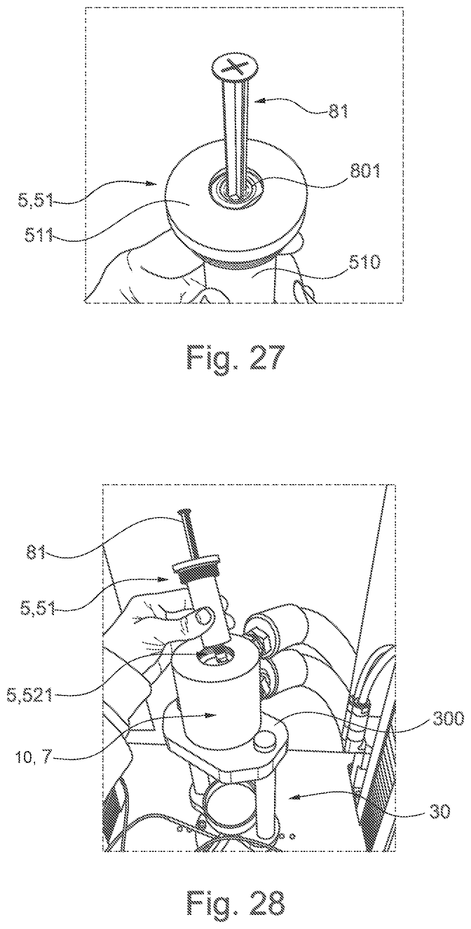

The threaded bush 521 is inserted into the through hole 512 of the RC 51 from the bottom. As can be seen in FIG. 25, the head 5211 of the threaded bush 521 faces downwards. The RC 51 with the threaded bush 521 can be inserted into the TC 7 as shown in FIG. 28. The syringe 8 can be inserted into the through hole 512 of the RC 51 as shown in FIG. 27 and the flange 801 of the syringe will rest on the upper side of the head 511 of the RC 51. The insert 5214 of the threaded bush 521 with the through hole 5212 formed therein, faces upwards. Thereby the NC 820 of the syringe 8 can enter the insert 5214.

With the second embodiment, the temperature of both the barrel 80 and the NC 820 of the syringe 8 can be adjusted. The temperature from the TC 7 will be transferred to the RC 51, wherein the barrel 80 is positioned. The RC 51 will transfer this temperature to the threaded bush 521 which is inserted into the RC 51 and thereby to the NC 820.

In FIG. 29 a third embodiment of the TD 1 is shown. This embodiment differs from the first and second embodiment of the TD 1 in that instead of a RC 51 a different AHE 5 is inserted into the tempering room 700 of the TC 7. In the third embodiment, the AHE 5 which is inserted into the TC 7 is a NH 52 in the shape of a disc body 520.

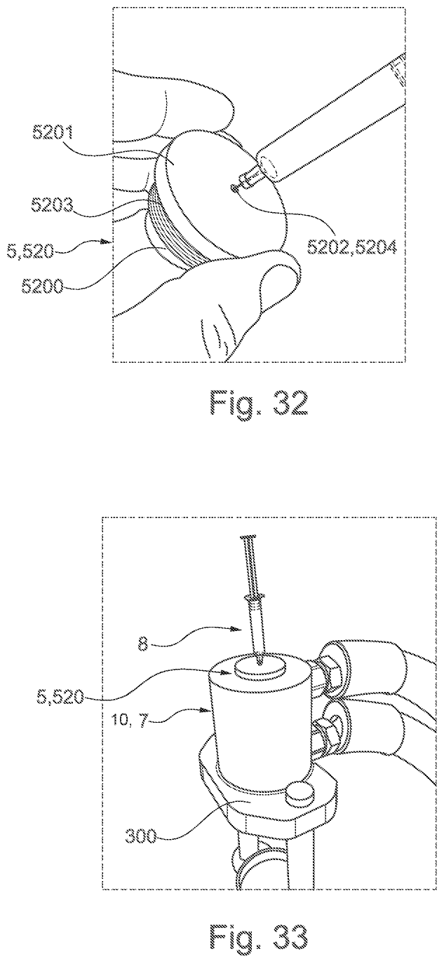

As can be best seen in FIG. 31, the disc body 520 has a head 5201 and a body 5200 extending from the bottom of the head 5201. The diameter of the head 5201 is larger than the outer diameter of the body 5200. In the upper section of the body 5200 adjacent to the head 5201 an outer thread 5203 is provided on the circumference of the body 5200. The disc body 520 has an axial through hole 5202 which extends over the entire height of the disc body 520. As can be derived the inner diameter of the through hole 5202 is smaller in the area of the head 5201 and upper section of the body 5200 and larger towards the lower end of the body 5200. The inner diameter of the through hole 5202 at the head 5201 of the disc body 520 corresponds to and preferably is equal to the outer diameter of the NC 820. A NC 820 inserted into the through hole 5202 is shown for example in FIG. 32.

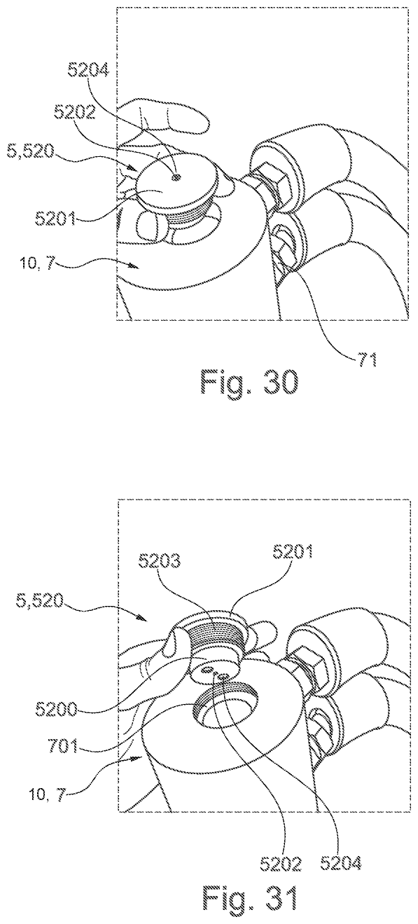

The disc body 520 is inserted into the tempering room 700 of the TC 7, which is a first IE 10, and is attached to the wall 70 by screw-type connection between the outer thread 5203 of the disc body 520 and the inner thread 701 of the TC 7. This is shown in FIGS. 30 and 31.

When inserted into the TC 7, the head 5201 of the disc body 520 extends over the upper surface of the TC 7, in particular the upper surface of the wall 70. This is for example depicted in FIG. 33. According to the third embodiment of the TD 1 a second IE 10, in particular a spacer 6 is provided over the disc body 520. The spacer 6 is formed by a cylindrical wall 60. At the bottom of the wall a receiving recess 61 is provided for receiving the head 5201 of the disc body 520. In the upper section of the wall 60 an inner thread 62 is provided at the inner circumference of the wall 60. Thereby, a FH 50 can be inserted into the upper end of the spacer 6. The FH 50 is a FH as described above with reference to the first embodiment of the fixture 3. The FH 50 is attached to the spacer 6 by screw-type connection between the outer thread 503 of the FH 50 and the inner thread 62 of the spacer 6. The spacer 6 may be made of a material of low heat transfer coefficient. For example, the spacer 6 may be made of plastic.

The spacer 6 has an inlet 64 and an outlet 65 for air. The inlet 64 and outlet 65 are provided in the wall 60 in the lower section. The inlet 64 may be connected to a tube 63 for input of airflow to the inside of the spacer 6. The inlet 64 and outlet 65 are arranged in such a distance from the bottom of the spacer 6, that they are at a height corresponding to the shoulder 803 of the syringe 8, which is inserted into the disc body 520. As can be seen in FIGS. 32 and 33, the syringe 8 is inserted into the disc body 520, in particular into the through hole 5202 of the disc body 520 only with the NC 820. Thereby, the temperature of only the NC 820 will be adjusted by the TC 7. The barrel 80 of the syringe is above the TC 7 and is thus not affected by the temperature set in the TC 7. As temperature convection may however occur from the upper side of the disc body 520 to the inside of the spacer 6, the air passing through the inlet 64 and outlet 65 is used to shield the barrel 80 from this temperature convection.

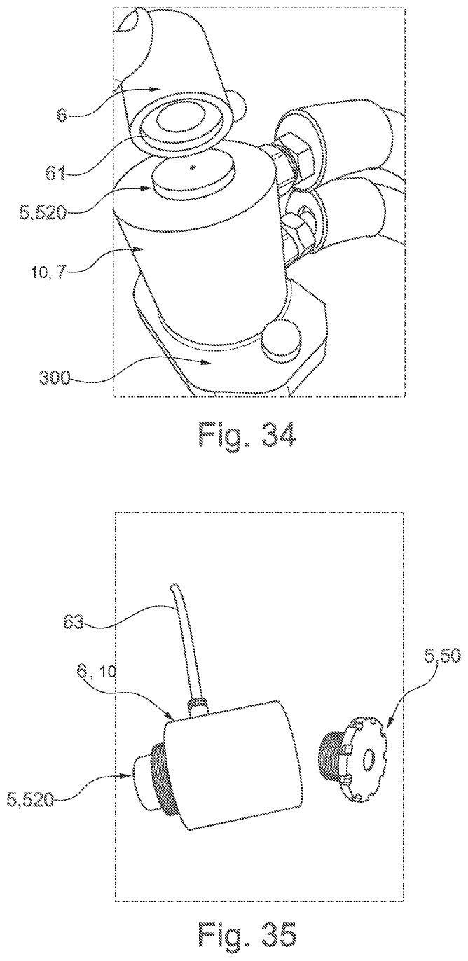

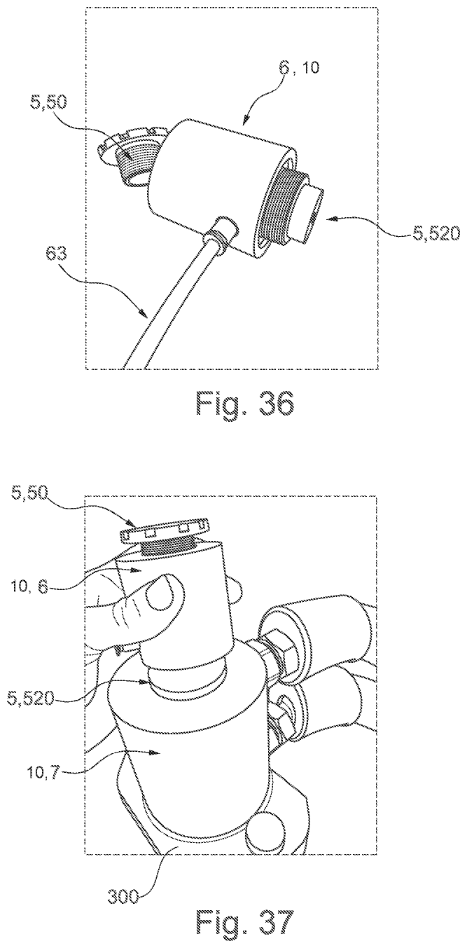

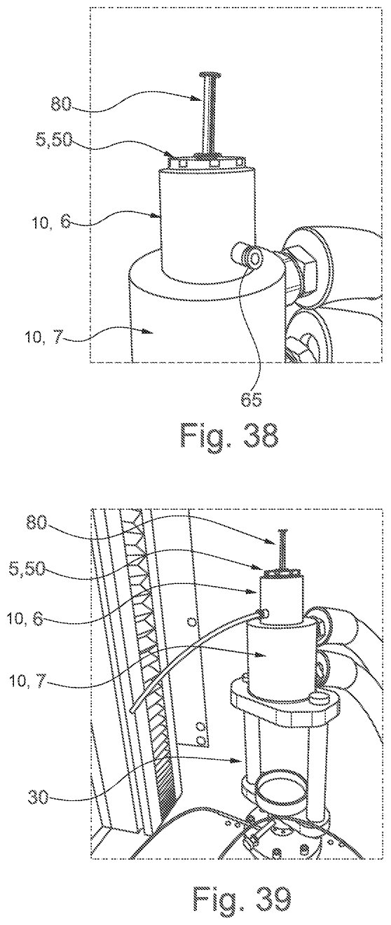

Once the spacer 6 is placed on the head 5201 of the disc body 520 and the FH 50 is screwed into the spacer 6 at the top, the syringe 8 can be inserted. The attachment of disc body 520 and FH 50 with the spacer 6 is shown in FIGS. 34 to 37. As shown in FIGS. 38 and 39 the syringe 8 is inserted with its barrel 80 into the FH 50 and the flange 801 rests on the collar 504 of the FH 50. In this position, the NC 820 is inside the through hole 5202 of the disc body 520. The through hole 5202 may be formed at least partially by an insert (not shown) corresponding to the insert 5214 of the threaded bush 521 described above.

Two measuring holes 5204 are provided in the bottom of the disc body 520 (see FIG. 29). In these measuring holes 5204 sensors 90, 91 as described above may be inserted from the bottom. The measuring holes 5204 extend from the bottom of the disc body 520 to a level close to the top of the head 5201 of the disc body 520.

The different embodiments of the fixture 3 and the TD 1 as described above can be realized with a fixture kit. One embodiment of such a fixture kit is shown in FIG. 9. The kit comprises the components of the SF 30, in particular a SP 300, a CP 302 and poles 301. In addition, the kit comprises at least two different AHEs 5. In the depicted embodiment, the AHEs are RCs 51, and NHs 52. The respective AHEs 5 thus differ in design. In addition, the AHEs differ in geometry. In particular, two NHs 52 in the form of disc bodies 520 are provided, wherein each disc body 520 has a through hole 5202 of a different diameter. These AHEs are used for the third embodiment of the TD1 as described above, i.e. for tempering the NC 820 of the syringe 8 only. Due to the different diameters of the through holes 5202, the appropriate NH 52 can be selected depending on the diameter of the NC 820 of the syringe 8 to be held. For example, NCs of sizes 25G or 27G may be tested.

The RCs 51 differ in that three are shown to have a wider diameter in the upper section of the through hole 512. These RCs 51 shown at the upper left of FIG. 9 are used for the first embodiment of the TD 1 described above, that means for tempering the barrel 80 of the syringe 8 only. The additional three RCs 51 shown at the upper right of FIG. 9 are RCs 51 with a continuous diameter of the through hole 512 and are adapted to receive a NH 52 in the form of a threaded bush 521 at its lower end. These RCs 51 are thus used for the second embodiment of the TD 1 as described above, i.e. for tempering both the NC 820 and the barrel 80 of the syringe.

The RCs 51 of each kind are provided in the kit with three different diameters of the through hole 512. Thereby, the appropriate RC 51 can be selected depending on the diameter of the syringe 8 to be held. For example, syringes with a volume of 0.5 ml, 1 ml or 2.25 ml may be tested.

In addition to the elements of the kit shown in FIG. 9, the kit can further comprise a spacer and at least one and preferably several FHs 50 with different diameter of the through hole 502.

The SP 300 shown in FIG. 9 is a SP 300 with a TC 7 affixed to it. The kit can further comprise a SP 300 as shown in FIG. 1 without TC 7 and with an aperture 3000 for receiving a FH 50.

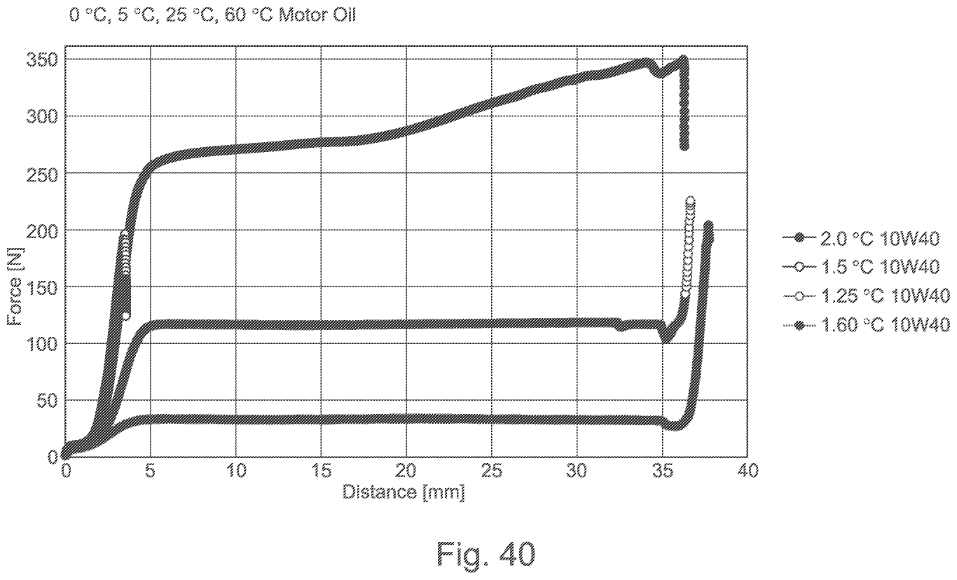

In FIGS. 40 to 43 graphs showing test results of moving force tests which were conducted with a TD 1 according to the invention are depicted.

In the test, the force required to empty a glass syringes at 100 mm/min was measured at 4 different temperatures, namely at: 0° C., 5° C., 25° C. and 60° C. The size of the NC of the syringes used was 29 G (Gauge). The waiting period between measurements was 10 min.

In the tests of FIG. 40, the syringe was filled with motor oil 10W40. As can be derived from FIG. 40 the force which was measured over the distance by which the plunger has moved, was higher for lower temperatures. The measured force is the moving force which is necessary to move the plunger.

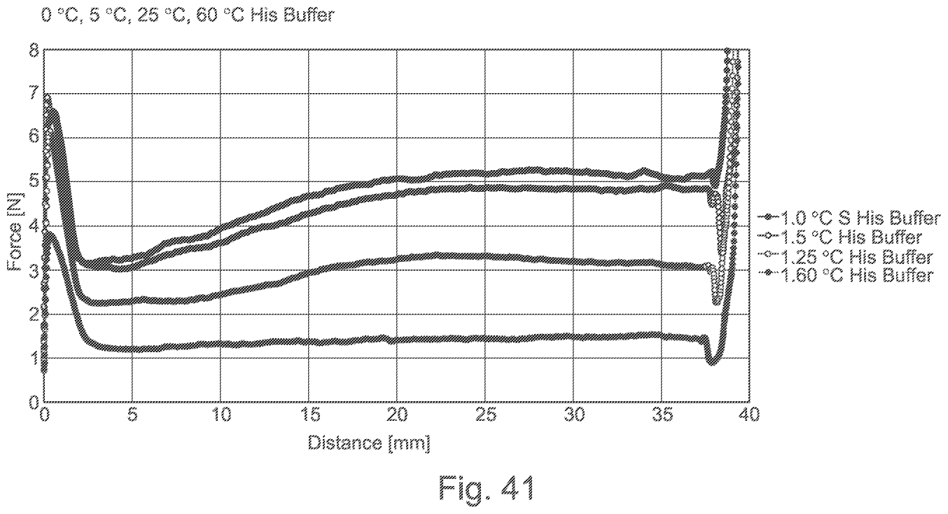

In the tests of FIG. 41, the syringe was filled with His buffer. The His buffer contained: 160 g of BSA (bovine serum albumin) 80 mg/ml, 6 g of PS80 (Polysorbate 80) 10%, 170 g of sucrose 85 mg/ml. His Buffer made of 3.104 g L-Histidine, 4.192 g Histidine Monohydrochloride, 2000 ml water and some HCl in order to reach a pH 6.0 was used to bring the final volume of the solution (2000 ml). Also in this graph it is obvious that the higher the temperature, the lower the required moving force.

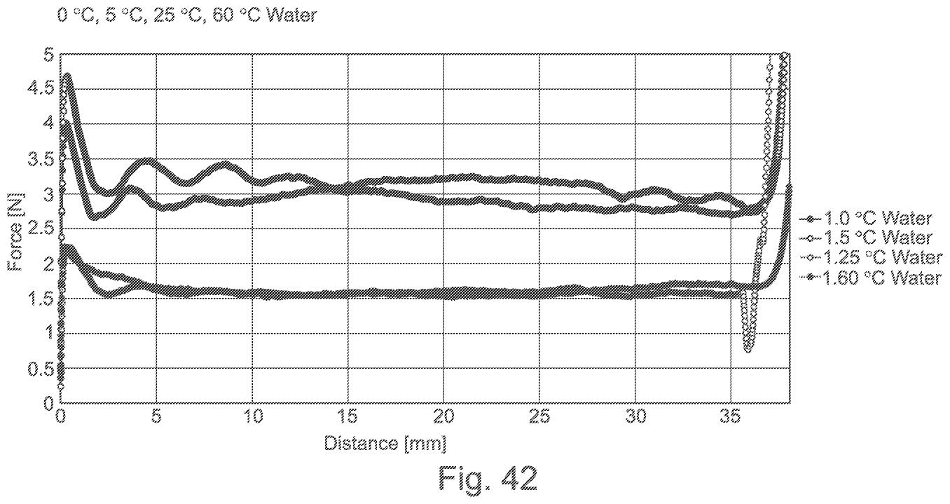

In the tests of FIG. 42, the syringe was filled with water. Also in this graph it is obvious that the higher the temperature, the lower the required moving force.

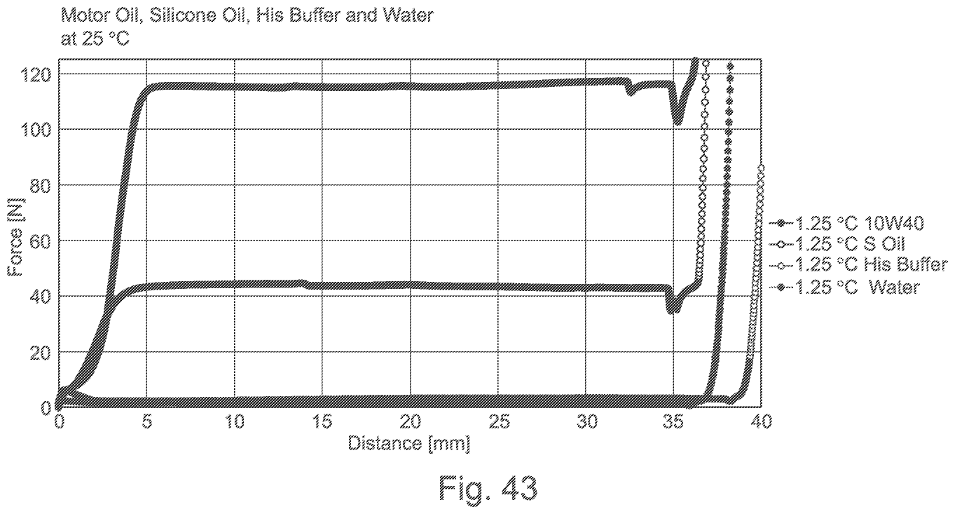

In FIG. 43 the test results for moving tests carried out at 25° C. with syringes filled with different liquids are compared. The difference of required force becomes obvious from this graph.

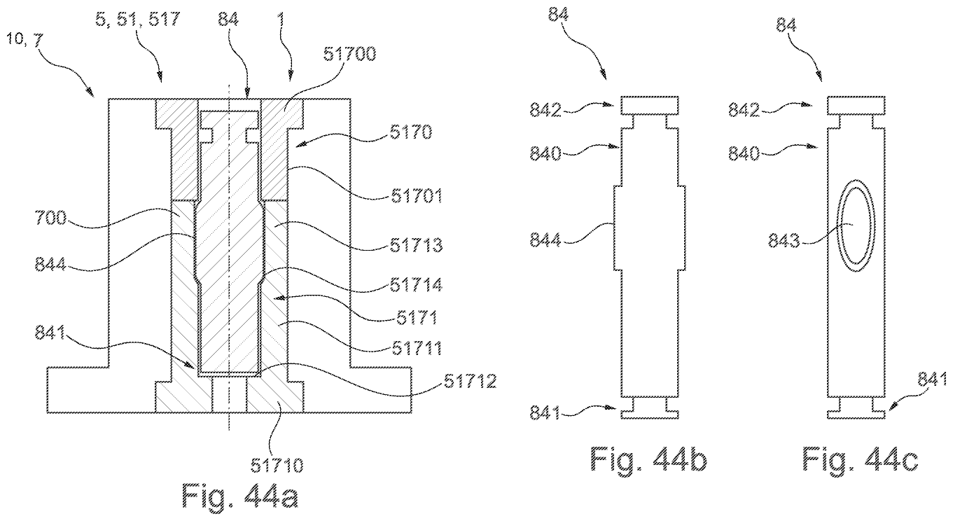

In FIG. 44a a schematic view of a fourth embodiment of the testing device 1 is shown. In FIG. 44a only the TC 7 and the AHEs 5 of the TD 1 are shown. This embodiment differs from the first embodiment by the layout of the RC 51. The RC 51 of the fourth embodiment differs from the RC 51 of the first embodiment as shown in FIGS. 10 to 19 in that the RC 51 according to the fourth embodiment is a two-part RC 517. The two-part RC 517 consists of an upper part 5170 and a lower part 5171. The upper part 5170 comprises a ring section 51700 and a tubular section 51701 extending from the bottom of the ring section 51700. The ring section 51700 has a larger outer diameter than the tubular section 51701. The ring section 51700 has an outer thread (not shown) for screw-type connection with the inner thread (not shown in FIG. 44a) of the TC 7. The tubular section 51701 extends into the tempering room 700 of the TC 7.

The lower part 5171 comprises a ring section 51710 and a tubular section 51711 extending from the top of the ring section 51710. The ring section 51710 has a larger outer diameter than the tubular section 51711. The ring section 51710 has an outer thread (not shown) for screw-type connection with an inner thread (not shown) in the lower part of the tempering chamber 700 of the TC 7. The tubular section 51711 extends into the tempering room 700 of the TC 7.

At the inner diameter of the lower part 5171 in a lower section, in particular in the vicinity or at the upper end of the ring section 51710, a step 51712 is provided. Thereby the inner diameter in the ring section 51710 is smaller than in the tubular section 51711. In the tubular section 51711 a tapered section 51714 is formed. This tapered section 51714 is preferably formed by two recesses 51713 which are formed in the inner diameter of the tubular section 51711 at diametrical opposite positions. The inner diameter of the tubular section 51711 in the angle range of the recesses 51713 is larger than the inner diameter of the tubular section 51701 of the upper part 5170. In the angle range where no recesses are provided, the inner diameter of the tubular section 51711 can be equal to the inner diameter of the upper part 5170. In the depicted embodiment the inner diameter of the tubular section 51701 and the inner diameter of the ring section 51700 of the upper part 5170 are equal.

The length of the tubular section 51701 of the upper part 5170 is shorter than the length of the tubular section 51711 of the lower part 5171.

In FIG. 44a it is shown that an auto-injector syringe 84 is inserted into the two-part RC 517.

One embodiment of the auto-injector syringe 84 is shown in FIGS. 44b and 44c. The auto-injector syringe 84 has a tubular casing 840 with a cap 841 movably received in the tubular casing 840 at the distal end of the tubular casing 840. The syringe is received in the tubular casing 840. In particular, the barrel, the plunger and the NC are surrounded by the tubular casing. At the top of the casing a head 842 is provided, which serves for activating the injection and dispensing the fluid from the syringe (not shown). The movable cap 841 is a safety mechanism. The cap 841 may have a though hole for the NC (not shown) or may be punctured by the NC. Once the cap 841 is moved proximally into the tubular casing 840, an activation mechanism (not shown) can be triggered by moving the head 842. The force necessary for moving the head 842 may be referred to as injection force.

In the outer circumference of the tubular casing 840 two windows 843 are provided. The windows 843 are formed in protrusions 844 which extend outwardly from the outer circumference of the tubular casing 840. The protrusions 844 are positioned on diametrically opposite sides of the tubular casing 840.

In the state which is shown in FIG. 44a, the cap 841 is pushed into the tubular casing 840 and the protrusions 844 are received in the recesses 51713 which form the tapered portion 51714 of the lower part 5171 of the two-part RC 517. The cap 841 rests on the step 51712 and the upper end of the protrusion 844 rests against the bottom end of the upper part 5170 of the two-part RC 517. In this state the auto-injector syringe 84 thus has been brought into the condition, where the safety mechanism provided by the cap 841 has already been overcome and the actual force necessary for injection, in particular dispensing fluid from the barrel (not shown) of the syringe can be measured.

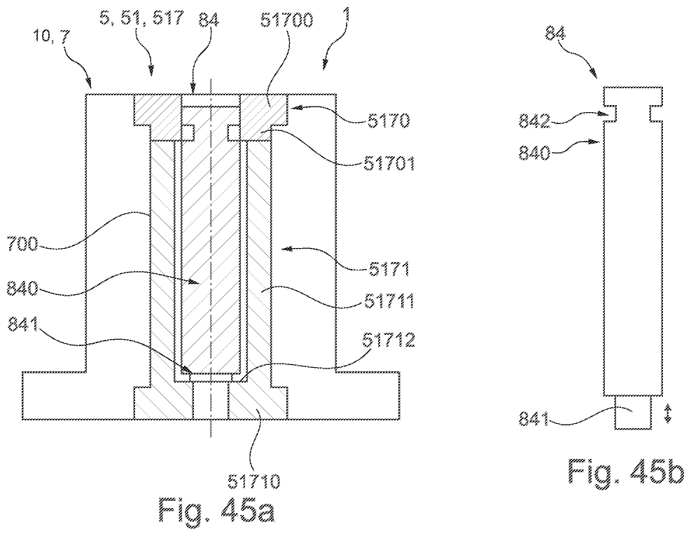

In FIG. 45a a schematic view of a fifth embodiment of the testing device 1 is shown. In FIG. 45a only the TC 7 and the AHEs 5 of the TD 1 are shown. This embodiment differs from the fourth embodiment by the layout of the RC 51. The RC 517 of the fifth embodiment differs from the RC 517 of the fourth embodiment in that the lower part 5171 is longer and does not have a tapered portion.

The embodiment of an auto-injector syringe 84 which is inserted into the two-part RC 517 in FIG. 45a is shown in FIG. 45b. As can be derived from FIG. 45b, the auto-injector syringe 84 only differs from the one shown in FIGS. 44b and 44c in that the cap 841 has a different shape and that no windows or protrusions are provided on the tubular casing 840 of the auto-injector syringe 84.

In the state as shown in FIG. 45a the cap 841 is pushed into the tubular casing 840. The cap 841 rests on the step 51712 and the upper end of the tubular casing 840 rests against the bottom end of the upper part 5170 of the two-part RC 517. In this state the auto-injector syringe 84 thus has been brought into the condition, where the safety mechanism provided by the cap 841 has already been overcome and the actual force necessary for injection, in particular dispensing fluid from the barrel (not shown) of the syringe can be measured.

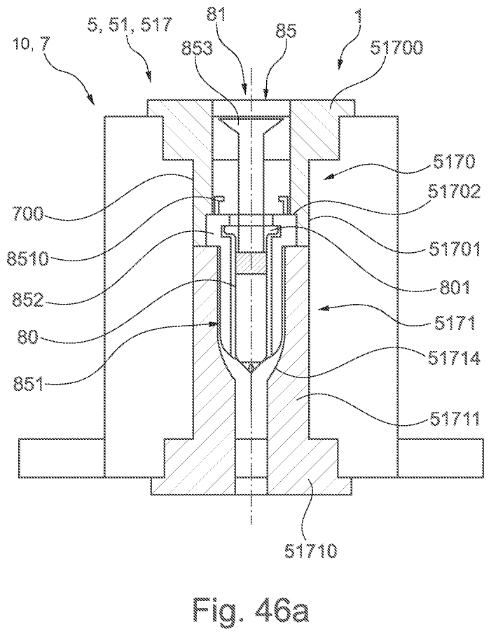

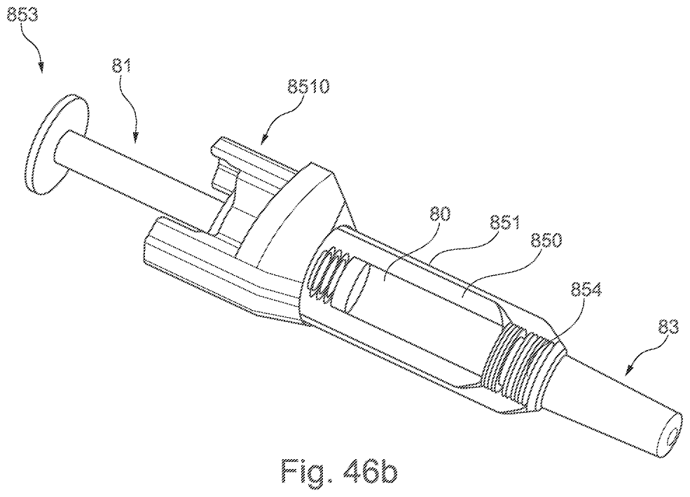

In FIG. 46a a schematic sectional view of a sixth embodiment of the testing device 1 is shown. In FIG. 46a only the TC 7 and the AHEs 5 of the TD 1 are shown. This embodiment differs from the fourth and fifth embodiment by the layout of the RC 51. The two-part RC 517 of the sixth embodiment differs from the RC 517 of the fourth and fifth embodiment in that the upper part 5170 has a step 51702 formed in its lower section and that the lower part 5171 does not have a step but has a tapered section 51714 at the inner diameter of the tubular section 51711.

The embodiment of a passive safety guard syringe 85 which is inserted into the two-part RC 517 is shown schematically in FIG. 46b. In this embodiment, the passive safety guard syringe 85 comprises a syringe 8 with a plunger 81. At the proximal end of the plunger 81 a plunger head 853 is provided. Inside of the shield 851 a syringe body 850 is provided. The syringe body encloses at least part of the barrel 80 of the syringe 8 and is attached to the syringe 8. At the proximal end the shield 851 has a flange 852. At the proximal side of the flange 852 latch members 8510 extend in the proximal direction. At the distal end of the shield 851, a spring 854 is provided inside the shield 851. In the state as shown in FIG. 46b, the spring 854 is compressed by the syringe body 850 or the syringe 8. The NC of the syringe is covered by a SCS 83.

In order to introduce such a passive guard syringe 85 into the RC 51 the lower part 5171 can then be introduced into the IE, in particular the TC 7, from the bottom. The lower part 5171 can be attached to the TC 7 by screw-type connection. Subsequently, the passive guard syringe 85 may be inserted into the lower part 5171 of the two-part RC 517 from the top after the SCS 83 has been removed. Subsequently, the upper part 5170 can be inserted into the tempering chamber 70 of the TC 7 from the top until step 51702 rests against the top of the flange 852 of the shield 851. The latch members 851 are positioned in the inner diameter of the upper section of the upper part 5170. The latch members 851 can be biased outwardly by the plunger head 8530 once the plunger 853 is pushed in distal direction. Thereby, an unlocking mechanism (not shown) can be activated and the syringe body 850 with the syringe 8 can be released from the shield 851 and can be pushed in a proximal direction by the spring 854. In this retracted position, the NC is enclosed in the shield 851 and the position of the syringe 8 relative to the shield 851 is preferably locked by a locking mechanism (not shown).

Thereby, the moving force for moving the plunger 81 in the barrel 80 as well as the force for activating the unlocking mechanism can be measured with the TD 1 in the embodiment as shown in FIG. 46a.

It should be noted, that the invention can also be used for passive safety guard syringes having unlocking mechanisms and locking mechanisms which are different from the ones described above. Also auto-injector syringes having different activation mechanisms than the ones described above can be used.

As the TD 1 and method of the invention allow testing of the moving force for moving a plunger of a syringe in the syringe at different conditions of the syringe, the invention can be used to for example to identify a recommendable administration temperature for a PFS. The invention can also be used to formulate pharmaceuticals such that the moving force at room temperature is in a convenient range. Furthermore, the invention allows for determining an appropriate geometry of the syringe, for example an appropriate diameter of the barrel or NC. The invention can also be used to determine the injection force and/or unlocking force of passive safety guard syringes or auto-injector syringes at different temperatures.

The invention thus provides a novel approach to characterize the drug delivery system flow performance dependent on single component temperature dependent contribution.

PFS and other combination products are receiving increasing attention as the container closure system of choice for injectable drug products where self-administration is preferable. Subcutaneous injections typically require the development of high protein concentration formulations, which results in several challenges for manufacturing, stability, analytical, and delivery.

Viscosity depends on many factors, including protein concentration, molecule properties, formulation and product temperature, and impacts injection force and dosing accuracy. Injection force depends on viscosity and product properties but also device components and their variability, such as inner needle diameter, container diameter, container lubrication and temperature at actual product usage.

Several standards and guidance documents require testing device functionality, including studies at upper and lower end temperatures. For example ISO 11608-1 delineates preconditioning temperatures for needle-based injection systems, the FDA guidance “technical considerations for pen, Jet, and related Injectors . . . ” requires the verification that injector performance is not adversely affected by environmental conditions such as “extreme conditions of use testing”.

It was poorly researched how and which component of the device contributes to an injection force change when modulating the device temperature (e.g. needle temperature vs. formulation temperature vs. lubrication temperature)

The invention provides a proprietary technology to selectively control and study the temperature of the syringe needle and the syringe barrel during injection force measurements to characterize the impact on injection force.

For example, 1 mL and 2 mL syringes with 25G, 27G and 29G needles and 2 different protein formulations can be tested at 2-8° C., 25° C. and 60° C. It has been identified that moving force, which corresponds to the injection force, and the device performance was significantly dependent on temperature, and that specific device components had different impact on the results.

### LIST OF REFERENCE NUMERALS

1 Testing device

10 Intermediate element

2 Load frame

20 base

21 post

22 connecting disk

3 Fixture

30 Support frame

300 Support plate

3000 aperture

3001 inner thread

3002 connecting hole

301 Poles

3010 Insulation ring

302 Carrier Plate

3020 Tray

303 Foot

5 Annular holding element

50 Flange holder

500 body

501 head

502 through hole

503 outer thread

504 collar

51 Receiving cylinder

510 body

511 head

512 through hole

513 outer thread

514 measuring hole

515 inner thread

516 step

517 two-part RC

5170 upper part

51700 ring section

51701 tubular section

51702 step

5171 lower part

51710 ring section

51711 tubular section

51712 step

51713 recess

51714 tapered section

52 Needle holder

520 Disc body

5200 body

5201 head

5202 through hole

5203 outer thread

5204 measuring hole

521 Threaded bush

5210 body

5211 head

5212 through hole

5213 outer thread

5214 insert

5215 recess

6 Spacer

60 wall

61 receiving recess

62 inner thread

63 Tube

64 inlet

65 outlet

7 Tempering cylinder

70 Wall

71 Connector

700 tempering room

701 inner thread

8 Syringe

80 barrel

800 lumen

801 flange

802 lumen

803 shoulder

804 neck

81 plunger

810 rod

811 stopper

82 dispensing opening

820 needle cannula

821 Luer cone

83 Syringe closing system

84 auto-injector syringe

840 tubular casing

841 cap

842 head

843 window

85 passive safety guard syringe

850 syringe body

851 shield

8510 latch member

852 flange

853 plunger head

854 spring

9 Sensing unit

90 Sensor

91 Sensor

92 Sensor

1. A testing device for testing the moving force of a plunger of a syringe, comprising a load frame and a fixture, FX, for holding a syringe in the testing device, TD, for moving force testing, wherein the fixture comprises an annular holding element, AHE, for holding part of the syringe, and a support frame, SF, having a support plate, SP, for supporting the annular holding element, AHE, wherein the annular holding element, AHE, is detachably connected to the support plate, SP, via an indirect connection via at least one intermediate element, IE, between the at least one annular holding element, AHE, and the support plate, SP, wherein one intermediate element, IE, is a tempering cylinder, TC, for tempering at least part of the syringe.

2. The testing device according to claim 1, wherein the at least one annular holding element, AHE, is a flange holder, FH, for holding the flange of the syringe.

3. The testing device according to claim 1, wherein the at least one annular holding element, AHE, is a needle holder, NH, for holding the needle cannula, NC, or Luer cone, LC, of the syringe.

4. The testing device according to claim 1, wherein the at least one annular holding element, AHE, is a receiving cylinder, RC, for receiving the barrel of the syringe to be held in the fixture, FX.

5. The testing device according to claim 1, wherein the fixture, FX, comprises at least two annular holding elements, AHEs, which are a flange holder, FH, and a needle holder, NH.

6. The testing device according to claim 1, wherein the fixture, FX, comprises at least three annular holding elements, AHEs, which are a flange holder, FH, a receiving cylinder, RC, and a needle holder, NH.

7. The testing device according to claim 1, wherein there is only one intermediate element, IE, wherein the only one intermediate element, IE, functions both as a tempering element and as a distance element.

8. The testing device according to claim 1, wherein there are two or more intermediate elements, IE, the function of at least one of the two ore more intermediate elements, IE, comprises the function of a tempering element; and the function of at least a second of the two or more intermediate elements, IE, comprises the function of a distance element.

9. Tho testing device according to claim 1, wherein there are two or more intermediate elements, IE, and at least one of the two or more intermediate elements, IE, is a tempering cylinder, TC; and at least a second of the two or more intermediate elements, IE, is a spacer.

10. The testing device according to claim 5, wherein the annular holding elements, AHEs, are directly attached to one another or indirectly attached via at least one intermediate element, IE, which is a spacer.

11. The testing device according to claim 1, wherein the tempering cylinder, TC, is adapted for receiving an annular holding element of the at least one annular holding element, AHE, in the form of a receiving cylinder, RC, for receiving the barrel of the syringe inserted into the inner diameter of the tempering cylinder, TC.

12. The testing device according to claim 1, wherein the tempering cylinder, TC, is adapted for receiving an annular holding element of the at least one annular holding element, AHE, which is a needle holder, NH, for receiving the needle cannula, NC, of the syringe and an annular holding element, AHE, which is a flange holder, FH, and the testing device, TD, comprises a spacer.

13. The device according to claim 1, wherein the testing device, TD, comprises at least two temperature sensors and the at least one annular holding element, AHE, has at least two measuring holes for insertion of a temperature sensor.

14. The testing device according to claim 1, wherein the load frame is a load frame for applying pressure on the plunger of a syringe held in the fixture of the testing device and for measuring the force for moving the plunger.

15. The testing device according to claim 1, wherein the at least one annular holding element, AHE, is made of metal.

16. A method for testing the moving force of a syringe, wherein the method is carried out with a testing device according to claim 1.

17. The method according to claim 16, wherein the method comprises the steps
a) setting the temperature in at least part of the syringe via the tempering cylinder TC,
b) applying a pressure on the plunger of the syringe via a load frame,
c) measuring the force for moving the plunger in the syringe via the load frame.

18. The method according to claim 17, wherein the method comprises step
d) monitoring the temperature present in at least part of the syringe via at least one temperature sensor.

19. The method according to claim 17, wherein the method comprises the step
e) monitoring the temperature in the vicinity of the barrel, in the vicinity of the needle cannula, NC, or Luer cone, LC, or at the inside of the barrel.

20. The method according to claim 17, wherein the method comprises the step
f) adjusting the temperature of at least part of the syringe.

21. A fixture kit for a fixture, FK, for holding a syringe in a testing device, TD for testing the moving force of a plunger of a syringe, comprising a load frame and the fixture, FX, for moving force testing, wherein the fixture comprises an annular holding element, AHE, for holding part of the syringe, and a support frame, SF, having a support plate, SP, for supporting the annular holding element, AHE, wherein the annular holding element, AHE, is detachably connected to the support plate, SP, via an indirect connection via at least one intermediate element, IE, between the at least one annular holding element, AHE, and the support plate, SP, wherein one intermediate element, IE, is a tempering cylinder, TC, for tempering at least part of the syringe wherein the fixture kit comprises the at least one support frame, SF, and the at least two annular holding elements, AHEs.

22. The fixture kit according to claim 21, wherein the fixture kit comprises the at least two annular holding elements, AHEs, wherein each annular holding elements, AHE, has an axial through hole and the inner diameter of the axial through hole of the at least two annular holding elements, AHEs, is different.

23. The Fixture kit according to claim 21, wherein the fixture kit comprises the tempering cylinder, TC.

24. The Fixture kit according to claim 23, wherein the TC is a TC for adjusting the temperature of a part of the syringe.
